SEQUENCE LISTING (16 sequences)

SEQ ID NO: 1 - protein, 343 aa, Artificial Sequence
  Description of Artificial SequenceModified Human Fibronectin Collagen-Binding Domain
MAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWLE

SEQ ID NO: 2 - protein, 159 aa, Artificial Sequence
  Description of Artificial SequenceHuman Basic Fibroblast Growth Factor with Enterokinase Recognition Sequence
DDDDKAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 58 aa, Artificial Sequence
  Description of Artificial SequenceHuman Epidermal Growth Factor with Enterokinase Recognition Sequence
DDDDKNSDSECPLSHDGYCLHDGVCMYIEALDKYACNCVVGYIGERCQYRDLKWWELR

SEQ ID NO: 4 - protein, 501 aa, Artificial Sequence [sequence omitted]

SEQ ID NO: 5 - protein, 400 aa, Artificial Sequence
  Description of Artificial SequenceHybrid Polypeptide of Human Fibronectin Collagen-Binding Domain and Human Epidermal Growth Factor
MAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWLDDDDKNSDSECPLSHDGYCLHDGVCMYIEALDKYACNCVVGYIGERCQYRDLKWWELR

SEQ ID NO: 6 - DNA, 49 bp, Artificial Sequence
  Description of Artificial SequencePCR Sense Primer for Human Fibronectin Collagen-Binding Domain
gaggtaccatggtacatatggcagctgtttaccaaccgcagcctcaccc

SEQ ID NO: 7 - DNA, 46 bp, Artificial Sequence
  Description of Artificial SequencePCR Antisense Primer for Human Fibronectin Collagen-Binding Domain
cgggatccttactcgagccactggatggggtgggagttgggctgac

SEQ ID NO: 8 - DNA, 1053 bp, Artificial Sequence
  Description of Artificial Sequence Modified Human Fibronectin Collagen-Binding Domain
ggtaccatggtacatatggcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggctcgagtaaggatcc

SEQ ID NO: 9 - DNA, 40 bp, Artificial Sequence
  Description of Artificial SequencePCR Sense Primer for Human Basic Fibroblast Growth Factor
gagtcgacgacgatgataaggcagccgggagcatcaccac

SEQ ID NO: 10 - DNA, 31 bp, Artificial Sequence
  Description of Artificial SequencePCR Antisense Primer for Human Basic Fibroblast Growth Factor
ggaattctcagctcttagcagacattggaag

SEQ ID NO: 11 - DNA, 489 bp, Artificial Sequence
  Description of Artificial SequenceHuman Basic Fibroblast Growth Factor with Enterokinase Recognition Sequence
gtcgacgacgatgataaggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagtcgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgagaattc

SEQ ID NO: 12 - DNA, 44 bp, Artificial Sequence
  Description of Artificial SequencePCR Sense Primer for Human Epidermal Growth Factor
gtgtcgacgacgatgataagaatagtgactctgaatgtcccctg

SEQ ID NO: 13 - DNA, 30 bp, Artificial Sequence
  Description of Artificial SequencePCR Antisense Primer for Human Epidermal Growth Factor
gaattcttagcgcagttcccaccacttcag

SEQ ID NO: 14 - DNA, 186 bp, Artificial Sequence
  Description of Artificial SequenceHuman Epidermal Growth Factor with Enterokinase Recognition Sequence
gtcgacgacgatgataagaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgctaagaattc

SEQ ID NO: 15 - DNA, 1527 bp, Artificial Sequence
  Description of Artificial SequenceHybrid Polypeptide of Human Fibronectin Collagen-Binding Domain and Human Fibroblast Growth Factor
ggtaccatggtacatatggcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggctcgacgacgatgataaggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagtcgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgagaattc

SEQ ID NO: 16 - DNA, 1224 bp, Artificial Sequence
  Description of Artificial SequenceHybrid Polypeptide of Human Fibronectin Collagen-Binding Domain and Human Epidermal growth factor
ggtaccatggtacatatggcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggctcgacgacgatgataagaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgctaagaattc

(19) United States
(12) Patent Application Publication
(10) Pub. No.: US 2002/0102709 A1
(43) Pub. Date: Aug. 1, 2002

(54) Collagen-binding physiologically active polypeptide

(76) Inventors: Tetsuya Ishikawa, Kanagawa (JP); Takashi Kitajima, Kanagawa (JP)

Correspondence Address: BURNS DOANE SWECKER & MATHIS L L P, POST OFFICE BOX 1404, ALEXANDRIA, VA, 22313-1404, US

(21) Appl. No.: 09/934,706

(22) Filed: Aug. 23, 2001

Related U.S. Application Data
(63) Continuation-in-part of application No. 09/507,691, filed on Feb. 22, 2000.

(30) Foreign Application Priority Data
Feb. 19, 1999 (JP) 11-041913
Nov. 1, 1999 (JP) 11-311364

(51) Int. Cl. C12N 9/64

(52) U.S. Cl. 435/226

(57) ABSTRACT

A collagen-binding physiologically active polypeptide is provided. In this polypeptide, a peptide from fibronectin is ligated to a physiologically active peptide, and this hybrid polypeptide is provided with both the collagen-binding activity and the physiological activity. A novel collagen matrix wherein the hybrid polypeptide is combined with collagen is also provided. The collagen-binding physiologically active polypeptide provided with both the collagen-binding activity and the physiological activity is useful as a drug delivery system (DDS) of the physiologically active peptide. Furthermore, this polypeptide can be combined with collagen to provide a functionally modified collagen matrix which is quite useful as a new biomaterial adapted for use in tissue regeneration.

### BACKGROUND OF THE INVENTION

This invention relates to a hybrid polypeptide, and more specifically, to a collagen-binding physiologically active polypeptide which is produced by connecting a peptide from fibronectin (FN) and a physiologically active peptide by a genetic engineering means and which has both the collagen-binding activity and the physiological activity; as well as a biomaterial produced by combining said collagen-binding physiologically active polypeptide with a polypeptide from collagen.

Cytokines, growth factors and other physiologically active peptides are expected for their use as a pharmaceutical. Basic fibroblast growth factor (bFGF) is an example of such polypeptides. It has a wide variety of biological activities including the activity of promoting proliferation and differentiation of various cells of mesodermal and neuroectodermal origin, that is, the activity of promoting cell growth, cell migration, and angiogenesis for vascular endothelial cells, fibroblasts, vascular smooth muscle cells, astroglia cells, or the like and bFGF is widely used as a growth factor for cell culture (Journal of Cell Biology, vol. 109, No. 1, pages 1 to 6 (1989)) In view of such activity, bFGF is highly expected for use as a wound healing agent (R. A. F. Clark ed., The Molecular and Cellular Biology of Wound Repair, s2nd ed., chapter 6, pages 237 to 248 (1988), Plenum, N.Y.). Epidermal growth factor (EGF) is also expected for its use as a wound healing agent in view of its cell-growth promoting activity for epithelial cells, vascular endothelial cells, and fibroblasts, and angiogeneic activity (Ziegler et al. ed., Growth Factors and Wound Healing, section 3, chapter 12, pages 206 to 228 (1997), Springer-Verlag, New York).

Physiologically active peptides, however, generally suffer from insufficient stability in the living tissues. Physiologically active peptides also suffer from the difficulty in realizing topical retention and sustained release in the target tissues, and as a consequence, likeliness of inducing side effects in other parts of the body. In view of such situation, practical use of many of such physiologically active peptides are deemed difficult, and there is a strong demand for an efficient drug delivery system (DDS) of such physiologically active peptides.

In order to solve the problem as described above, many attempts have been made to provide the physiologically active peptide with a sustained release capability by mixing the peptide with an appropriate biocompatible high-molecular weight material. Among such materials, collagen is a protein which has been widely used in the field of medicine as a biomaterial for repairing tissue injury and as a pharmaceutical carrier because of its excellent biocompatibility.

A technique for controlling the slow release property of a physiologically active peptide by mixing the peptide with collagen is disclosed, for example, in Japanese Patent No. 2641755 and JP-A 5-43453.

JP-B 4-18865 discloses a collagen matrix containing a growth factor produced by mixing the growth factor with the collagen followed by thermal dehydration and cross-linking.

In the process disclosed in JP-A 8-253429 and JP-A 8-325160, sustained release matrices are prepared by mixing heparin, a physiologically active peptide which has heparin-binding activity, and collagen for the purpose of providing the physiologically active peptide with the sustained release capability. Such approach wherein the physiologically active peptide is used with heparin, however, suffers from the problem of anticoagulant action inherent to the heparin as well as the limitation of inapplicability to physiologically active peptides having no heparin-binding activity.

Fujioka et al. (Fujioka et al., Journal of Controlled Release vol. 33, pages 307 to 315 (1995)) has reported an improvement in stability and topical retention of interferon by mixing the interferon with collagen of high co S ncentration, and embedding the air-dried collagen pellet containing the interferon in the living tissues.

Anti-thrombogenic materials prepared by direct chemical cross-linking of heparin with collagen are also reported (Raghunath et al., Journal of Biomedical Materials Research, vol. 17, pages 613 to 621 (1983), and Nimni et al., Journal of Biomedical Materials Research, vol. 21, pages 741 to 771 (1987)).

Another attempt that has been made is chemical cross-linking of the physiologically active peptide with collagen by glutaraldehyde or the like.

As described above, there is a strong demand for an efficient DDS for many types of physiologically active peptides, and collagen has been expected as a promising carrier capable of improving the sustained release property of the physiologically active peptide. Affinity of the physiologically active peptide for collagen, however, is generally low, and stable retention of the physiologically active peptide in the collagen matrix for prolonged period has so far failed by merely mixing the peptide with the collagen.

The approach of chemical cross-linking of the physiologically active peptide with the collagen has also suffered from the problem of loss or reduction of the physiological activity.

In the more recent techniques developed in view of such situation, the physiologically active peptide is connected with other peptides by means of genetic engineering to thereby realize targeting of the physiologically active peptide. To be more specific, expression of a hybrid polypeptide in Escherichia coli (E. coli) or the like has been enabled by the genetic recombination technology. The expressed hybrid polypeptide, however, does not necessarily exhibit its original activity, and it is often the case that a difficulty is encountered in expressing the polypeptide that exhibits the desired property, and in particular, the desired physiological activity.

JP-A 5-178897 discloses a method for producing a functional polypeptide by ligating bFGF with the cell-binding domain sequence of fibronectin (FN), which is a component of extracellular matrix. This functional polypeptide binds to the cell to exhibit the cell-growth promoting activity. However, topical retention or sustained release is not realized due to the direct binding of the polypeptide to the cells. JP-A 5-178897 also describes significantly reduced bFGF activity of the functional polypeptide.

Another attempt for realizing the sustained release through the extracellular matrix is collagen targeting TGF-β which is a hybrid polypeptide of collagen-binding decapaptide from bovine von Willebrand factor and transforming growth factor β (TGF-β) (U.S. Pat. No. 5,800,811, Tuan et al., Connective Tissue Research, vol. 34, No. 1, pages 1 to 9 (1996), Han et al., Protein Expression and Purification, vol. 11, pages 169 to 178 (1997), and Gordon et al., Human Gene Therapy, vol. 8, pages 1385 to 1394 (1997)).

These reports, however, showed that most of the recombinant proteins recovered from inclusion body in E. coli exhibited impaired collagen-binding activity as well as reduced physiological activity. Furthermore, only a small proportion of the hybrid polypeptide exhibited collagen-binding activity but not TGF-β growth activity after its binding to collagen although the polypeptide succeeded in capturing the cells. Accordingly, the objects of topical retention and sustained release were not realized. In view of such situation, the collagen-binding decapeptide from von Willebrand factor is inappropriate as the collagen-binding peptide for the production of a collagen-binding physiologically active polypeptide.

In the meanwhile, Nishi et al. produced collagen-binding growth factors which were hybrid polypeptides of bFGF or EGF with the collagen-binding polypeptide from collagenase of Clostridium histolyticum, which is an anaerobic Gram-negative bacillus. However, Nishi et al. reported that the collagen-binding activity of the collagen-binding growth factors produced in their attempt was insufficient and their product failed to bind to the culture dishes coated with collagen as well as to many types of collagen materials (Proc. Natl. Acad. Sci. USA, vol. 95, pages 7018 to 7023 (1998)). Of course, such collagen-binding growth factors can not exhibit cell-growth promoting activity after its binding to collagen, since they cannot bind to the collagen materials. The collagen-binding growth factors were also found to experience decrease in their EGF activity, and from immunological point of view, a polypeptide from a bacterial collagenase is apparently inadequate for use in human tissue regeneration.

In reducing the production cost in the industrial production of the hybrid polypeptide by genetic engineering, it is quite important to realize the production of polypeptide in prokaryotes (for example, bacteria) or in yeast, and in particular, expression of the polypeptide in E. coli.

Expression in E. coli, however, is often associated with the difficulty in reproducing the original activity of the polypeptide or producing the desired polypeptide at a high yield.

A major problem in the expression in E. coli is difficulty in obtaining the soluble polypeptide of sufficient amount. In other words, the polypeptide is likely to be produced in an insoluble form of the inclusion body, or to be decomposed immediately after its synthesis, and these are believed to be the consequences of the exclusion mechanism of E. coli since the expression of a large amount of an exogenous polypeptide is a stress to E. coli. The mechanism of the inclusion body formation is not yet fully understood, and there is no almighty solution for such inclusion body formation. An effective means is solubilization of the inclusion body using a strong protein-denaturing agent followed by renaturation of the recombinant protein. This process, however, is associated with the drawback that the three dimensional structure of the polypeptide is not finally reproduced, and the resulting polypeptide does not exhibit the normal physiological activity.

Various methods are known in the art which can be used for producing the hybrid polypeptide of various types and which is capable of efficiently producing the soluble protein with the activity maintained, and wherein the protein can be renatured even if the inclusion body is formed. Known polypeptide partners for the production of the hybrid polypeptide include thioredoxin, protein A, chloramphenicol acetyl transferase, galactose-binding protein, maltose-binding protein (MBP), glutathione S-transferase (GST), and β-galactosidase, and expression vector for many of these polypeptide partners are already commercially available.

Production of a soluble hybrid polypeptide with the activity retained is likely to be realized by the use of such polypeptide partner at a relatively high probability. However, none of the above-mentioned polypeptide partners exhibit collagen-binding activity. Use of a polypeptide partner other than those mentioned above is likely to result in a low probability of the hybrid polypeptide expression as well as even lower probability of producing a protein having the activity maintained. In summary, feasibility of the production of an active hybrid polypeptide by using a partner other than those described above is proved only after demonstrating both the production of the hybrid polypeptide using the particular polypeptide partner, and the activity of the hybrid polypeptide product.

The collagen-binding domain of FN may be postulated as a collagen (gelatin)-binding polypeptide partner.

For example, use of a polypeptide from FN as a tag in the purification of a recombinant protein of interest is proposed. To be more specific, a vector capable of expressing a polypeptide including the gelatin-binding domain of FN connected to the desired protein, and the method of purifying the desired protein are disclosed in U.S. Pat. No. 5,342,762 and U.S. Pat. No. 5,460,955, and a polypeptide comprising the collagen-binding domain of FN connected to other polypeptide or a therapeutic agent, and its purification method are proposed in JP-A 62-89699.

In U.S. Pat. No. 5,342,762 and U.S. Pat. No. 5,460,955, supra, the gelatin-binding domain of rat FN with defined sequence is claimed, and the hybrid polypeptide comprising the FN-derived part and the protein of interest is captured by utilizing affinity of the FN-derived part (purification tag) to the collagen/gelatin, and the desired protein is recovered by cleavage with a specific protease.

To be more specific, prepro-sequence or leader signal peptide sequence of FN which is an amino acid sequence of 32 amino acid residues which promotes secretion of the recombinant polypeptide from an insect cell or an animal cell to the medium is added at the amino terminal of the gelatin-binding domain of FN, and the production and the purification are conducted in the insect cell using a baculovirus expression system as described in the Examples, or the production and the purification is conducted in the animal cell such as COS cell used as the host cell.

More specifically, trypsin cleavage site is located on the amino terminal side of the gelatin-binding domain of FN, and the desired protein is located on the amino terminal side of the trypsin cleavage site. Factor XIIIa site is located on the amino terminal side of the desired protein and the prepro-sequence or the leader signal peptide sequence is added on the amino terminal side of the factor XIIIa site.

However, these US patents are utterly silent about the presence/absence of the physiological activity of the part other than the FN-derived part, namely, the part of the desired protein in the hybrid polypeptide.

The proteins of interest disclosed in the embodiments of the expression and purification of such proteins include homology unit I-12 (12th type I homology unit), homology unit I-9 (9th type I homology unit), and homology unit III-1 (1st type III homology unit) of FN as well as various parts of human factor IX. These polypeptides are not physiologically active in the sense of the present invention.

As a reflection of such situation, these US patents are also silent about the activity, function, and applicability of the hybrid polypeptides even if the polypeptide of interest happened to be a physiologically active peptide. Applications as the agent promoting proliferation of human cells, tissue regeneration, and organ regeneration are not at all indicated, and even if such application had been indicated (which is not), the hybrid polypeptide of the constitution as described above is inappropriate for such use.

To be more specific, in the polypeptide of these US patents wherein the protein of interest is connected on the amino terminal side of the gelatin-binding domain of FN, the trypsin cleavage site is included at the junction in the resulting polypeptide, and even if the trypsin cleavage sequence was replaced with another protease recognition sequence, at least one unnecessary amino acid residue derived from the protease recognition sequence is left in the protein of interest after the cleavage by the protease. As a consequence, lost or decrease of the physiological activity of the protein of interest will take place.

When purification is the only purpose of the use of the FN-derived part, such part may be removed by cleavage with trypsin or other endoprotease followed by decomposition with carboxypeptidase B or the like to thereby obtain the protein of interest which is free from any unnecessary sequence. With no such treatment, the protein after the endoprotease cleavage will include excessive amino sequence on its carboxyl terminal, and the protein will exhibit either reduced or no activity. Accordingly, the process of these US patents are unsuitable for the case wherein the resulting hybrid polypeptide is used for tissue regeneration or pharmaceuticals.

In addition, when the hybrid polypeptide of the constitution as described above is produced in E. coli or other prokaryotes, the hybrid polypeptide experiences decrease in collagen-binding ability, decrease or loss of the physiological activity of the protein of interest will take place. Also, such hybridpolypeptide will exhibit extremely low or no recovery of the active polypeptide in water-soluble form from the inclusion body.

In other words, production of a polypeptide of animal origin with retaining the function is relatively easy by using an animal cell or an insect cell for the host, while the production of such polypeptide in prokaryotes (bacteria or the like) or yeast is associated with an extreme difficulty. However, the production of a genetically engineered polypeptide in a prokaryote such as E. coli is extremely advantageous in reducing the production cost although such production is very difficult and many breakthroughs are required for the realization.

JP-A 62-89699, supra discloses a sequence including the polypeptide constituted from the amino acid sequence of human FN from Thr³⁷⁹ to Val⁴⁴⁵. The polypeptide disclosed in JP-A 62-89699 is unsuitable as a partner of a hybridpolypeptide. As will be described later, the sequence used in the Examples of JP-A 62-89699 does not correspond to a sequence obtained by protease cleavage. The hybrid polypeptide of such sequence and a physiologically active peptide exhibited substantially no collagen-binding activity, and substantially no or reduced physiological activity.

JP-A 8-140677 proposes a technique for targeting a functional protein in the extracellular matrix. In this technique, a chimera protein is produced, and in this protein, an amino acid sequence from the exogenous protein or peptide is inserted between 70 kDa fragment at the N terminal of the FN molecule and 37 kDa fragment at the C terminal of the FN molecule. Practice of this invention will encounter two main obstacles. This technique is unsuitable for industrial production using E. coli or the like in view of the high molecular weight and insolubility in water of the chimera protein. To exhibit the function, the chimera protein is necessary to be expressed in animal cells, and its utility is likely to be limited to gene therapy wherein the gene of the chimera protein is directly expressed in the human cell.

### SUMMARY OF THE INVENTION

As described above, production of a hybrid polypeptide having the activity of sufficient level is difficult in spite of some proposals that have been made wherein a hybrid polypeptide is expressed in E. coli or the like by utilizing genetic recombination technique.

In view of the situation as described above, an object of the present invention is to provide a collagen-binding physiologically active polypeptide wherein activity of the physiologically active peptide is maintained at a sufficient level and the collagen-binding activity is imparted by genetic engineering means. Another object of the invention is to realize topical retention and prolonged, controlled slow release of the physiologically active polypeptide by providing a biomaterial (a physiologically active polypeptide/collagen composite) produced by combining the collagen-binding physiologically active polypeptide as described above with a polypeptide from collagen

In order to obviate the situation as described above, the inventors of the present invention found that a collagen-binding physiologically active polypeptide which is provided with both the activity of the physiologically active peptide and the collagen-binding activity can be produced by ligating the collagen-binding domain of fibronectin (FN) with the physiologically active peptide by genetic engineering means. The inventors of the present invention also found that a biomaterial (a physiologically active polypeptide/collagen composite) can be produced by combining the collagen-binding physiologically active polypeptide with a polypeptide from collagen. The present invention has been accomplished on the bases of such findings. The objects of the present invention are achieved by (1) to (20) as described below.

(1) A collagen-binding physiologically active polypeptide having both collagen-binding activity and physiological activity different from fibronectin-activities including collagen-binding activity comprising:

a first peptide having collagen-binding activity and consisting of an amino acid sequence which is identical to an amino acid sequence of protease-hydrolysis fragment of fibronectin with at least one protease selected from a group consisting of trypsin, chymotrypsin, thermolysin, plasmin, thrombin, cathepsin D, cathepsin G, pepsin, subtilisin, leukocyte elastase and chymase, and which corresponds to an internal amino acid sequence in collagen-binding domain ranging from 28 kDa to 75 kDa from the amino-terminal of fibronectin,

fused with

a second peptide having physiological activity different from fibronectin activities including collagen-binding activity.

(2) A collagen-binding physiologically active polypeptide according to the above (1) wherein said first peptide consists of an internal amino acid sequence of human fibronectin selected from a group consisting of

from Ala²⁶⁰ to Trp⁵⁹⁹ (Ala² to Trp³⁴ of SEQ ID NO.1),

from Ala²⁶⁰ to Leu⁴⁸³ (Ala² to Leu ²²⁵ of SEQ ID NO.1),

from Ala²⁶⁰ to Arg⁴⁸⁴ (Ala² to Arg ²²⁶ of SEQ ID NO.1),

from Val²⁶² to Arg⁴⁸⁴ (Val⁴ to Arg ²²⁶ of SEQ ID NO.1),

from Val²⁶² to Trp⁵⁹⁹ (Val⁴ to Trp ³⁴¹ of SEQ ID NO.1),

from Val³⁷⁷ to Leu⁴⁸³ (Val¹¹⁹ to Leu²²⁵ of SEQ ID NO.1),

from Val³⁷⁷ to Trp⁵⁹⁹ (Val ¹¹⁹ to Trp ³⁴¹ of SEQ ID NO. 1)

from Leu⁴⁸³ to Trp⁵⁹⁹ (Leu²²⁵ to Trp ³⁴¹ of SEQ ID NO.1),

from Arg⁴⁸⁴ to Trp⁵⁹⁹ (Arg ²²⁶ to Trp ³⁴¹ of SEQ ID NO.1).

from Ala²⁶¹ to Phe⁵⁸⁴ (Ala³ to Phe³²⁶ of SEQ ID NO.1),

from Ala²⁶¹ to Gln⁴⁸² (Ala³ to Gln²²⁴ of SEQ ID NO. 1)

from Arg⁴⁸⁴ to Phe⁵⁸⁴ (Arg²²⁶ to Phe³²⁶ of SEQ ID NO.1)

from Val²⁶² to Phe⁵⁸⁴ (Val ⁴ to Phe³²⁶ of SEQ ID NO.1)

from Leu⁴⁸³ to Phe⁵⁸⁴ (Leu²²⁵ to Phe³²⁶ of SEQ ID NO. 1)

and from Asp⁴⁸⁵ to Trp⁵⁹⁹ (Asp ²²⁷ to Trp ³⁴¹ of SEQ ID NO.1).

(3) A collagen-binding physiologically active polypeptide according to the above (1) wherein said protease-hydrolysis is a proteolysis with a combination of chymotrypsin and plasmin.

(4) A collagen-binding physiologically active polypeptide according to the above (3) wherein the first peptide consists of an amino acid sequence of human fibronectin Ala²⁶⁰ to Trp⁵⁹⁹ (Ala² to Trp³⁴¹ of SEQ ID NO.1).

(5) A collagen-binding physiologically active polypeptide according to the above (1) wherein said protease-hydrolysis is a proteolysis with trypsin.

(6) A collagen-binding physiologically active polypeptide according to the above (5) wherein the first peptide consists an amino acid sequence Ala²⁶⁰ to Arg⁴⁸⁴ (Ala² to Arg²²⁶ of SEQ ID NO. 1)

(7) A collagen-binding physiologically active polypeptide according to the above (1) wherein said protease-hydrolysis is a proteolysis with a combination of trypsin and chymotrypsin.

(8) A collagen-binding physiologically active polypeptide according to the above (7) wherein the first peptide consists of an amino acid sequence Asp⁴⁸⁵ to Trp⁵⁹⁹ (Asp²²⁷ to Trp³⁴¹ of SEQ ID NO.1).

(9) A collagen-binding physiologically active polypeptide according to any one of the avobe (1) to (8) wherein said second peptide is a physiologically active peptide selected from a group consisting of a cytokine, insulin, parathyroid hormone and matrix metalloproteinases(MMPs).

(10) A collagen-binding physiologically active polypeptide according to any one of the avobe (1) to (9) wherein said second peptide is a cytokine.

A collagen-binding physiologically active polypeptide according to the above (10) wherein said polypeptide remains bound to collagen or is released gradually from collagen with plasma fibronectin in order to stimulate cells.

(11) A collagen-binding physiologically active polypeptide according to the above (10) wherein said cytokine is a growth factor.

(12) A collagen-binding physiologically active polypeptide according to any one of the avobe (1) to (11) wherein said second peptide is fused on the carboxyl terminal side of said first peptide.

(13) A collagen-binding physiologically active polypeptide according to the above (12) wherein an amino acid spacer having less than 7 residues is inserted at the fusion site of the first peptide.

(14) A collagen-binding physiologically active polypeptide according to the above (13) wherein a carboxyl terminal of said amino acid spacer is a proteolytic site.

(15) A collagen-binding physiologically active polypeptide according to any one of the avobe (1) to (14) wherein said collagen-binding activity is inhibited competitively by fibronectin.

(16) A collagen-binding physiologically active polypeptide according to any one of the avobe (1) to (15) wherein said polypeptide is produced in bacteria.

A collagen-binding physiologically active polypeptide according to the above (16) wherein said polypeptide exhibits its physiological activity on collagen unless competed with plasma fibronectin, and wherein said polypeptide exhibits its physiologically activity when released gradually in plasma.

(17) A collagen-binding physiologically active polypeptide according to any one of the avobe (1) to (16) wherein said polypeptide is produced in a transformant containing a recombinant vector including the gene coding for said collagen-binding physiologically active polypeptide.

Further, A collagen-binding physiologically active polypeptide according to any one of the avobe (1) to (17) wherein said polypeptide exhibits its physiological activity after binding to collagen either in a collagen-bound state or by being slowly released from collagen.

A collagen-binding physiologically active polypeptide according to any one of the avobe (1) to (17) wherein said polypeptide is released from collagen with plasma fibronectin to perform tissue regeneration.

A collagen-binding physiologically active polypeptide according to any one of the avobe (1) to (17) wherein said polypeptide is produced by genetic engineering technique using a bacterial host and has the ability to be solubilized in water.

(18) An agent for enabling topical retention or sustained release of a physiologically active peptide or a physiological activity-imparting agent which contains the collagen-binding physiologically active polypeptide of any one of the avobe (1) to (17).

(19) A biomaterial comprising a composite wherein the collagen-binding physiologically active polypeptide of any one of the above (1) to (17) is combined with collagen or gelatin.

(20) An agent for enabling topical retention or sustained release of a physiologically active peptide or a physiological activity-imparting agent which contains the biomaterial of the above (19).

Further, A gene coding for the collagen-binding physiologically active polypeptide of any one of the above (1) to (17).

A recombinant vector including the gene coding for the collagen-binding physiologically active polypeptide of claim any one of the above (1) to (17).

A recombinant vector including the gene coding for the collagen-binding physiologically active polypeptide of any one of the above (1) to (17).

A bacteria including the gene coding for the collagen-binding physiologically active polypeptide of any one of the above (1) to (17).

A transformant containing a recombinant vector including the gene coding for the collagen-binding physiologically active polypeptide of any one of the above (1) to (17).

A transformant including the gene coding for the collagen-binding physiologically active polypeptide of any one of the above (1) to (17).

A transformant containing a recombinant vector including the gene coding for the collagen-binding physiologically active polypeptide of any one of the above (1) to (17).

A bacteria containing a recombinant vector including the gene coding for the collagen-binding physiologically active polypeptide of any one of the above (1) to (17).

Escherichia coli (E. coli) containing a recombinant vector including the gene coding for the collagen-binding physiologically active polypeptide of any one of the above (1) to (17).

A method for producing the collagen-binding physiologically active polypeptide of any one of the above (1) to (17) by using Escherichia coli (E. coli).

### BRIEF DESCRIPTION OF THE DRAWINGS

FIG. 1 schematically depicts the collagen-binding physiologically active polypeptide.

FIG. 2 schematically depicts the physiologically active polypeptide/collagen matrix composite.

FIG. 3 schematically depicts the batch process wherein gelatin-binding activity of the FNCBD-FGF is evaluated.

FIG. 4 shows the SDS-PAGE gel of the supernatant obtained in the batch process.

FIG. 5 schematically depicts ELISA wherein collagen-binding activity is evaluated.

FIG. 6 is a graph showing the collagen-binding activity (in ELISA) of FNCBD.

FIG. 7 is a graph showing the collagen-binding activity (in ELISA) of FNCBD-FGF.

FIG. 8 is a graph showing the collagen-binding activity (in ELISA) of FNCBD-EGF.

FIG. 9 is a graph showing growth-promoting activity of FNCBD-EGF for BALB/c3T3 cell.

FIG. 10 is a graph showing growth-promoting activity of FNCBD-FGF for human fibroblast.

FIG. 11 is a graph showing growth-promoting activity of FNCBD-EGF for NRK49F cell.

FIG. 12 schematically depicts the function of the collagen-binding physiologically active polypeptide.

FIG. 13 is a graph showing human keratinocyte growth-promoting activity of the FNCBD-EGF/collagen composite.

FIG. 14 is a graph showing the comparison of the binding activity to various collagen types between the FNCBD-EGF and the EGF.

FIG. 15 is a graph showing the long term stability of the cell-growth promoting activity of the FNCBD-EGF/collagen composite.

FIG. 16 is a graph showing the collagen targeting activity in a high-concentration protein solution of the FNCBD-EGF in comparison with that of the EGF.

FIG. 17 is a graph showing the release or the sustained release of the FNCBD-EGF by human plasma FN.

FIG. 18 is a graph showing the collagen-binding activity (in ELISA) of FN485-599/EGF.

FIG. 19 is a graph showing the cell growth-promoting activity of FN485-599/EGF.

FIG. 20 is a graph showing the collagen-binding activity (in ELISA) of FN260-484/EGF.

FIG. 21 is a graph showing the cell growth-promoting activity of FN260-484/EGF.

### PREFERRED EMBODIMENTS OF THE INVENTION

Fibronectin (FN) is a cell-adhesive glycoprotein found in plasma and extracellular matrix as well as on the surface of the cultivated cells. Fibronectin is known to be capable of binding to high molecular weight biomolecules such as collagen (gelatin), heparin, fibrin, and integrin, and to be involved in cell adhesion, tissue organization, repairing of the injured tissue, and other biological processes (Ruoslahti, Annual Review of Biochemistry, vol. 57, pages 375 to 413 (1988)).

The collagen-binding physiologically active polypeptide of the present invention is a polypeptide wherein the peptide from FN as described above is ligated to a physiologically active peptide, and the polypeptide of the present invention exhibits both the collagen-binding activity and the physiological activity and the physiological activity is maintained after binding to the collagen. In other words, the collagen-binding physiologically active polypeptide of the present invention is a functional hybridpolypeptide wherein the peptide from FN is ligated to the physiologically active peptide by genetic engineering means.

It should be noted that the conventional investigations as described above failed to provide a hybrid polypeptide, which had both the collagen-binding activity and the physiological activity. In practical point of view, physiological activity of the hybrid polypeptide after binding to collagen is particularly important, which was not demonstrated by the conventional hybrid polypeptide.

The inventors of the present invention have also attempted to produce hybrid polypeptides of FGF or EGF and the collagen-binding peptide from human von Willebrand factor. This attempt, however, was abandoned since the resulting hybrid polypeptide exhibited insufficient collagen-binding activity as well as insufficient yield.

The inventors of the present invention have conducted further investigation and found that the collagen-binding activity and the physiological activity can be maintained at a sufficient level when the collagen-binding peptide from FN is used for the collagen-binding polypeptide, and that the activity of the physiologically active polypeptide is maintained even after the binding of the hybrid polypeptide to the collagen. On the bases of such finding, the inventors of the present invention invented the collagen-binding physiologically active polypeptide which is a novel functional hybrid polypeptide as well as its application.

The present invention has also enabled to impart the collagen-binding activity based on the peptide from FN to the physiologically active polypeptide which has no collagen-binding activity as well as the physiologically active peptide which inherently has the collagen-binding activity.

The term “collagen” used in the present invention includes collagen as well as thermally denatured collagen such as gelatin. Therefore, the term “gelatin-binding” activity is substantially equivalent to “collagen-binding” activity, and in the present invention, the collagen-binding ability may be depicted as the gelatin-binding ability and vice versa.

The collagen-binding activity of the peptide derived from FN is quite stable and high in the hybrid polypeptide, and such stable collagen-binding activity has enabled to complete a physiologically active polypeptide/collagen composite which is a physiologically active biomaterial wherein the physiologically active moiety is retained in the collagen matrix or from which the physiologically active moiety is gradually released.

Furthermore, the collagen-binding physiologically active polypeptide of the present invention has the nature that its collagen-binding activity is competitively inhibited by the plasma FN. In other words, the collagen-binding physiologically active polypeptide of the present invention is released from the collagen when it is exposed to plasma, serum, or blood, or in the co-presence or upon addition of the plasma, serum, or blood, while it is retained in collagen in scarcity of the plasma, serum, or blood.

As described above, it has been critical in completing the present invention that the collagen-binding peptide derived from FN was used for the collagen-binding part in the production of the collagen-binding physiologically active polypeptide.

In the state of the recombinant DNA technology and the genetic engineering technology of today, hybrid gene (fusion gene) can be produced from the gene of the physiologically active peptide, in principle, by ligating the gene of the peptide from any part of the FN. However an adequate selection of the collagen-binding peptide sequence is critical for the production of a hybrid polypeptide wherein both the collagen-binding and the physiological activities are maintained. Such selection is also within the scope of the present invention.

<Peptide from FN (Fibronectin Collagen-binding Domain, FNCBD)>

As described above, the peptide derived from FN which is a critical component in the present invention is a collagen-binding peptide derived from FN, and preferably, a sequence corresponding to a collagen-binding peptide produced by proteolysis of FN using a protease, and more preferably, by limited proteolysis or naturally occurred proteolysis of the FN.

To be more specific, the peptide derived from FN may have an amino acid sequence which is completely homologous with the amino acid sequence of the polypeptide constituting the collagen-binding domain of FN which has the collagen and/or gelatin-binding activity; an amino acid sequence wherein one to several amino acid residues has been added to such amino acid sequence; or an amino acid sequence where in one to several amino acid residues has been substituted, deleted, or added in such amino acid sequence.

Preferred examples of such proteases include trypsin, chymotrypsin, thermolysin, plasmin, thrombin, cathepsin D, and pepsin, which may be used alone or in combination of two or more.

Preferably, FN is human FN. Use of FN from other animal species is also acceptable in some cases.

In view of the situation as described above, the term “FN collagen-binding domain (FNCBD)” used in the present invention is a collagen/gelatin-binding polypeptide from FN which is located between the position about 28 kDa from the amino terminal of FN to the position about 75 kDa from the amino terminal of FN, and which is obtained by limited hydrolysis of FN by trypsin, chymotrypsin, thermolysin, plasmin, thrombin, cathepsin D, cathepsin G, pepsin, subtilisin, chymase, leucocyte elastase, or a combination of such proteases.

Exemplary polypeptides include collagen or gelatin-binding polypeptide of about 30 kDa obtained by limited hydrolysis with trypsin or limited hydrolysis with subtilisin; collagen or gelatin-binding polypeptide of about 39 to 45 kDa obtained by limited hydrolysis with chymotrypsin, limited hydrolysis with cathepsin and thrombin, limited hydrolysis with leucocyte elastase, limited hydrolysis with thermolysin, or limited hydrolysis with chymase; and polypeptide of human FN from Ala²⁶⁰ to Trp⁵⁹⁹ obtained by limited hydrolysis with plasmin and chymotrypsin.

Exemplary collagen-binding FN polypeptides also include polypeptides having the sequence of amino acid sequence which constitutes the polypeptide of human FN from Ala²⁶⁰ to Trp⁵⁹⁹ polypeptides having a sequence which is homologous to such sequence; and polypeptides having such sequence wherein a part of the sequence is substituted or deleted or wherein another sequence is inserted or added. Examples of such human FN peptide which may be used in the present invention are those having the following sequences:

(1) the amino acid sequence of the polypeptide of human FN from Ala²⁶⁰ to Trp⁵⁹⁹ obtained from human FN by limited hydrolysis with a protease or natural hydrolysis and which has collagen/gelatin-binding activity; an amino acid sequence which is homologous to such sequence; or an amino acid sequence wherein a part of the sequence is substituted or deleted or wherein another sequence is inserted or added;

(2) the amino acid sequence which constitutes the polypeptide of human FN from Ala²⁶⁰ to Trp⁵⁹⁹; an amino acid sequence which is homologous to such sequence; or an amino acid sequence wherein a part of the sequence is substituted or deleted or wherein another sequence is inserted or added; wherein a protease recognition sequence is present on the carboxyl terminal of said amino acid sequence, and said protease recognition sequence is the one from human FN;

(3) an amino acid sequence including all of the amino acid sequence which constitutes the polypeptide of human FN from Ala²⁶⁰ to Trp⁵⁹⁹; an amino acid sequence which is homologous to such sequence; or an amino acid sequence wherein a part of the sequence is substituted or deleted or wherein another sequence is inserted or added; and

(4) an amino acid sequence which constitutes a polypeptide obtained by limited hydrolysis with a protease or natural hydrolysis of the polypeptide of human FN from Ala²⁶⁰ to Trp⁵⁹⁹ and which has collagen/gelatin-binding activity; an amino acid sequence which is homologous to such sequence; or an amino acid sequence wherein a part of the sequence is substituted or deleted or wherein another sequence is inserted or added.

Retention of the gelatin-binding activity in the hybrid polypeptide prepared by using FN peptide for the polypeptide partner can be relatively easily confirmed by measuring the gelatin-binding activity of the polypeptide obtained by limited hydrolysis with protease of the hybrid polypeptide comprising the polypeptide of human FN from Ala²⁶⁰ to Trp⁵⁹⁹ and the physiologically active peptide.

The degree of the gelatin-binding activity may be measured by evaluating the retention of the gelatin binding in the presence of high concentration salt, for example, 2M NaCl, or in an experiment wherein competitive inhibition with plasma FN is evaluated.

It should be noted that there are exceptional cases wherein use of a sequence which is slightly different from the amino acid sequence which is homologous to the amino acid sequence constituting the gelatin-binding polypeptide from FN obtained from human FN by limited hydrolysis with a protease or natural hydrolysis does not result in the loss of the collagen-binding activity of the resulting hybrid polypeptide.

However, it should be noted that, in most cases, the hybrid polypeptide wherein a FN amino acid sequence which can be obtained only by genetic engineering process wherein cleavage site in the protease hydrolysis or the natural hydrolysis is utterly disregarded is used as the FN-derived part for fusion with the physiologically active peptide exhibits significantly reduced collagen-binding activity even if the FN-derived part used was a sequence covering all or part of the FN collagen-binding domain. For example, the hybrid polypeptides as disclosed in JP-A 62-89699 wherein the polypeptide comprising the amino acid sequence of human FN from Cys ²⁷⁷ to Ser ⁵⁷⁷ or the polypeptide comprising the amino acid sequence of human FN from Thr ³⁷⁹ to Val ⁴⁴⁵ is fused to a physiologically active peptide by genetic engineering means suffer from greatly impaired collagen/gelatin-binding activity. The hybrid polypeptides produced by fusing the human FN polypeptide from Thr ³¹⁴ to Met ⁴⁴⁶, the human FN polypeptide from Thr ³⁷⁴ to Gly ⁵⁰⁷, the human FN polypeptide from Thr ³⁷⁴ to Gly ⁵⁰⁷, or the like, or the polypeptide comprising the human FN amino acid sequence from Thr³⁷⁴ to Met ⁴⁴⁶ reported in the article published by the inventors of JP-A 62-89699 (Owens et al., EMBO J, vol. 5, pages 2825 to 2830 (1986)) with a physiologically active peptide by genetic engineering means also exhibit greatly reduced gelatin-binding activity.

In view of such situation, when the hybrid collagen-binding physiologically active peptide is prepared by genetic engineering process, the sequence of the collagen-binding peptide from FN should be prepared by selecting a sequence homologous to the gelatin-binding peptide which is produced by limited hydrolysis with a protease or natural hydrolysis.

The cleavage site by the protease is C terminal side of arginine and lysine in the case of trypsin; C terminal side of isoleucine, leucine, phenylalanine, tyrosine, tryptophan and threonine in the case of chymotrypsin; C terminal side of isoleucine, leucine, valine, phenylalanine, and methionine in the case of thermolysin; C terminal side of arginine and lysine in the case of plasmin; between arginine and glycine in the case of thrombin; C terminal side of lysine, tyrosine, phenylalanine and arginine in the case of cathepsin D; C terminal side of leucine, phenylalanine, tyrosine, and methionine in the case of pepsin; C terminal side of 2 to 3 consecutive amino acids having short side chain such as Gly, Ala and Val in the case of leucocyte elastase; C terminal side of leucine, tyrosine, phenylalanine in the case of cathepsin G; and C terminal side of alanine in the case of subtilisin.

In the actual hydrolysis of FN, and in particular, in the limited hydrolysis of FN, there are sites which are likely to be cleaved and the sites which are resistant to the cleavage because of the higher order structure of FN. In other words, the number of the collagen/gelatin-binding peptides obtained by the limited hydrolysis with the protease is limited.

Therefore, when the collagen-binding peptide domain is used in the production by genetic engineering technique, a site which is more susceptible to a protease must be adopted as the site of the division.

To be more specific, the site in FN which is susceptible to protease cleavage and the gelatin-binding activity of the cleaved domain may be investigated by examining the articles in the past (Ruoslahti et al., J. Biol. Chem., vol. 254, pages 6054-6059 (1979), Balian et al., J Biol Chem., vol. 254, pages 1429-32 (1979), Ruoslahti et al., J. Biol. Chem., vol. 254, pages 6054-6059 (1979), Hahn et al., Proc. Natl. Acad. Sci., vol. 76, pages 1160-1163 (1979), Gold et al., Proc. Natl. Acad. Sci. U S A., vol. 76, pages 4803-7 (1979), Furie et al., J Biol Chem., vol. 255, pages 4391-4 (1980), Engvall et al., Coll. Relat. Res., vol. 1, pages 505-516 (1981), McDonald et al., J. Biol. Chem., vol. 256, pages 5583-7 (1981), Vartio, T et al., J. Biol. Chem., vol. 256, pages 471-7 (1981), De Petro, G et al., Proc. Natl. Acad. Sci. U S A., vol. 78, pages 4965-9 (1981), Ruoslahti et al., J. Biol. Chem., vol. 256, pages 7277-81 (1981), Vartio et al., Eur. J. Biochem., vol. 123, pages 223-33 (1982), Petersen et al., Proc. Natl. Acad. Sci., vol. 80, pages 137-141 (1983), Skorstengaard et al., Eur. J. Biochem., vol. 140,pages 235-243 (1984), Zardi et al., Eur. J. Biochem., vol. 146, pages 571-579(1985), Skorstengaard et al., Eur. J. Biochem., vol. 161, pages 441-453 (1986), and the like), or by actually conducting the limited hydrolysis of FN by protease and examining the amino terminal, the carboxyl terminal, or the amino acid sequence of the gelatin-binding peptide.

Although considerable number of studies have been conducted on the collagen-binding peptide of FN, there is discrepancy between the studies (Owens et al., EMBO J., vol. 5, pages 2825 to 2830 (1986), Ingham et al., J. Biol. Chem, vol. 264, pages 16977 to 16980 (1989), Litvinovich et al., J. Mol. Biol., vol. 217, pages 563 to 575 (1991), Banyai et al., Eur. J. Bioche, vol. 193, pages 801 to 806 (1990), Skorstengaard et al., FEBS letters, vol. 343, pages 47 to 50 (1994)).

It is the view of the inventors of the present invention that such discrepancy between the studies is caused by the conclusion without considering the difference between the peptide obtained by genetically engineering means (the sequence divided at an inadequate site) and the peptide obtained by protease cleavage.

Selection of the sequence divided at an inadequate site and addition of purification tag in the production of the FN-derived part from FN by genetic engineering means are the causes for the loss or decrease of the collagen-binding activity even if the particular region has the collagen-binding activity inherent to the polypeptide from FN. Moreover, in the case of a hybrid polypeptide, the collagen/gelatin-binding activity of the polypeptide from FN is often lost by the fusion even in the case wherein such activity had been present in the polypeptide from FN before its fusion. It is likely that the original higher order structure of the polypeptides is distorted by the inadequate adoption of the FN-derived peptide to invite the loss of activity in the fusion polypepeptide.

The collagen-binding physiologically active peptide of the present invention is produced by the fusion of the physiologically active peptide with the collagen-binding peptide from FN which has been divided at an appropriate site and which has no purification tag added thereto. In addition, the collagen-binding physiologically active polypeptide of the present invention is a recombinant hybrid peptide produced in E. coli wherein the collagen/gelatin-binding activity of FN is substantially retained to enable binding with gelatin, and therefore, the polypeptide of the present invention is highly adapted for gelatin affinity purification. The collagen-binding physiologically active peptide of the present invention also exhibits highly-maintained physiological activity of the original physiologically active peptide.

It should be noted that the collagen-binding peptide sequence from FN is not the only collagen-binding peptide sequences reported in the art, and other sequences that have been reported include sequences of collagenase and gelatinase which are matrix metalloproteinases, and von Willebrand factor, decorin, biglycan, fibromodulin, osteonectin, vitronectin, thrombospondin and the like in the extracellular matrix. However, no hybrid polypeptide has been reported to have the function equivalent to those of the present invention, and the attempts conducted by selecting the collagen-binding peptide from the bacterial collagenase or the von Willebrand factor as mentioned above for the collagen-binding polypeptide are no exceptions.

Preferred examples of the polypeptide from human FN for use in the production of the collagen-binding physiologically active polypeptide (hybrid polypeptide) in the present invention are not limited to those covering the full length of the polypeptide constituted by the amino acid sequence of human FN from Cys ²⁷⁷ to Ser ⁵⁷⁷ (the sequence described in JP-A 62-89699).

Examples of such polypeptides include:

polypeptide consisting of the amino acid sequence of human FN from Val ²⁶² to Arg ⁴⁸⁴ obtained by limited hydrolysis with subtilisin and trypsin;

polypeptide consisting of the amino acid sequence of human FN from Ala ²⁶⁰ to Leu ⁴⁸⁴ obtained by limited hydrolysis by plasmin, chymotrypsin and pepsin;

polypeptide consisting of the amino acid sequence of human FN from Ala ²⁶⁰ to Arg ⁴⁸⁴ obtained by limited hydrolysis by trypsin;

polypeptide consisting of the amino acid sequence of human FN from Val³⁷⁷ to Leu ⁴⁸³ obtained by limited hydrolysis by thermolysin and pepsin;

polypeptide consisting of the amino acid sequence of human FN from Val ³⁷⁷ to Trp ⁵⁹⁹ obtained by limited hydrolysis by plasmin, chymotrypsin and pepsin;

polypeptide consisting of the amino acid sequence in human FN Leu⁴⁸³ to Trp⁵⁹⁹obtained by limited hydrolysis by plasmin, chymotrypsin and thermolysin;

polypeptide consisting of the amino acid sequence of human FN from Asp ⁴⁸⁵ to Trp ⁵⁹⁹ obtained by limited hydrolysis by trypsin and chymotrypsin;

polypeptide consisting of the amino acid sequence of human FN from Ala²⁶¹ to Phe ⁵⁸⁴ obtained by limited hydrolysis by thermolysin;

polypeptide consisting of the amino acid sequence of human FN from Ala²⁶¹ to Gln ⁴⁸² obtained by limited hydrolysis by thermolysin;

polypeptide consisting of the amino acid sequence of human FN from Arg⁴⁸⁴ to Phe ⁵⁸⁴ obtained by limited hydrolysis by thermolysin and pepsin;

polypeptide consisting of the amino acid sequence of human FN from Val²⁶² to Phe ⁵⁸⁴ obtained by limited hydrolysis by thermolysin;

polypeptide consisting of the amino acid sequence of human FN from Leu⁴⁸³ to Phe 584 obtained by limited hydrolysis by thermolysin; and

polypeptide consisting of the amino acid sequence of human FN from Arg⁴⁸⁴ to Trp ⁵⁹⁹ obtained by limited hydrolysis by pepsin and chymotrypsin.

Preferred examples of the polypeptide from FN for use in the production of the collagen-binding physiologically active polypeptide in the present invention also include those covering the full length of the polypeptide constituted by the amino acid sequence of human FN from Cys ²⁷⁷ to Ser ⁵⁷⁷. Such examples include:

the polypeptide of human FN from Ala ²⁶⁰ to Trp ⁵⁹⁹ obtained by limited hydrolysis with plasmin and chymotrypsin, and the polypeptide of human FN from Val²⁶² to Trp ⁵⁹⁹ obtained by limited hydrolysis with subtilisin and chymotrypsin.

It should be noted that many polypeptides which include the entire length of the polypeptide sequence constituted from the human FN amino acid sequence from Cys ²⁷⁷ to Ser ⁵⁷⁷ are inadequate for use as the FN-derived collagen-binding polypeptide of the collagen-binding physiologically active polypeptide. A complete list of such inadequate polypeptides will be huge, and a part of the list includes polypeptides constituted from:

amino acid sequence of human FN from Asn 220 to Ser ⁵⁷⁷;

amino acid sequence of human FN from Arg ²²¹ to Ser ⁵⁷⁷;

amino acid sequence of human FN from Gly ²²² to Ser ⁵⁷⁷,

amino acid sequence of human FN from Asn 223 to Ser ⁵⁷⁷,

amino acid sequence of human FN from Leu ²²⁴ to Ser ⁵⁷⁷;

amino acid sequence of human FN from Leu ²²⁵ to Ser ⁵⁷⁷;

amino acid sequence of human FN from Gln ²²⁶ to Ser ⁵⁷⁷,

amino acid sequence of human FN from Cys ²²⁷ to Ser ⁵⁷⁷,

amino acid sequence of human FN from Ile ²²⁸ to Ser ⁵⁷⁷;

amino acid sequence of human FN from Cys ²⁷⁷ to Ser ⁵⁷⁷;

amino acid sequence of human FN from Cys ²⁷⁷ to Cys ¹²⁰¹;

amino acid sequence of human FN from Cys ²⁷⁷ to Thr ¹²⁰²;

amino acid sequence of human FN from Cys ²⁷⁷to Phe ¹²⁰³;

amino acid sequence of human FN from Cys ²⁷⁷ to Asp ¹²⁰⁴;

amino acid sequence of human FN from Cys ²⁷⁷ to Asn ¹²⁰⁵;

amino acid sequence of human FN from Cys ²⁷⁷ to Leu ¹²⁰⁶;

amino acid sequence of human FN from Cys ²⁷⁷ to Ser ^{1207v};

amino acid sequence of human FN from Cys ²⁷⁷to Pro ¹²⁰⁸; and

amino acid sequence of human FN from Cys ²⁷⁷to Gly ¹²⁰⁹;

As described above, coverage of the full length of the amino acid sequence of human FN from Cys ²⁷⁷ to Ser ⁵⁷⁷ (the sequence described in JP-A 62-89699) is not sufficient in the selection of the amino acid sequence of the polypeptide from FN for the production of the collagen-binding physiologically active peptide of the present invention. The amino acid sequence from Thr ³⁷⁹ to Val ⁴⁴⁵ was included in both cases wherein the use of the FN-derived part was adequate and inadequate, and therefore, coverage of the full length of such region is not the sufficient condition for the polypeptide from FN used in the collagen-binding physiologically active peptide of the present invention. JP-A 62-89699 described the region of Thr ³⁷⁹ to Val ⁴⁴⁵ as a minimal sequence having the collagen-binding ability. However, the polypeptide constituted from the amino acid sequence from The 374 to Ala ⁴⁷⁹ covering the region of Thr ³⁷⁹ to Val ⁴⁴⁵ was subsequently reported to have no collagen-binding ability even when the sequence was not fused with other sequence (Skorstengaard et al., FEBS letters, vol. 343, pages 47 to 50 (1994)).

<Physiologically Active Peptide>

The term “physiologically active peptide” used in the present invention is a generic designation of the peptides and the polypeptides having various physiological activities including cytokines and growth factors. Exemplary physiologically active peptides include growth factors such as fibroblast growth factor (FGF) family, transforming growth factor β (TGF-β) family, epidermal growth factor (EGF) family, platelet-derived growth factor (PDGF), insulin-like growth factors (IGFs), nerve growth factor (NGF), vascular endothelial growth factor (VEGF), hepatocyte growth factor (HGF) and other growth factors, and cell differentiation factors such as bone-morphogenetic proteins (BMPs); cytokines such as interferons (IFNs), interleukins (ILs), colony-stimulatingfactors (CSFs), erythropoietin, and tumor necrosis factor (TNF); hormons such as insulin and parathyroid hormone (PTH); enzymes such as matrix metalloproteinases(MMPs) and other proteases. It should be noted that the peptides sequences widely used in the field of genetic engineering as a fusion partner or a purification tag such as maltose-binding protein (MBP), glutathione S-transferase (GST), histidine tag, and β-galactosidase are not included in the present invention in the concept of the physiologically active peptides.

The term “physiological activity” or “activity of the physiologically active peptide” used in the present invention designates a part or all of the activity of the “physiologically active peptidel” as defined above. For example, in the case of a cytokine or a growth factor, modulating activities of cellular functions such as growth, differentiation, migration, synthesis of biological substances, and the like are included within the scope of the physiological activity while mere binding to the cell, receptor, or the like is not included in the concept of physiological activity. In the present invention, the activities inherent to FN are also outside the scope of physiological activity.

In the present invention, function as a fusion partner or a purification tag, for example, an activity of binding to a particular substance are also excluded from the concept of physiological activity. Exemplary such activities are affinity for metal chelates of thioredoxin, IgG-binding activity of protein A, chloramphenicol-binding activity of chloramphenicol acetyl transferase, galactose-binding ability of galactose-binding protein, amylose-binding activity of MBP, glutathione-binding activity of GST, nickel-binding activity of histidine tag, a part or all of the enzymatic activities of β-galactosidase.

The binding activities or the enzymatic activities of such fusion partners or the purification tags are already demonstrated to be maintained in various hybrid peptides, and the activity of the polypeptide fused with such fusion partner or purification tag is also known to be well-preserved in the hybrid peptides. Such activities, therefore, should be regarded as exceptional.

<Hybrid Polypeptide>

In the present invention, the fibronectin collagen-binding domain (FNCBD) and the physiologically active peptide are ligated by genetic engineering means, and the physiologically active peptide is preferably ligated on the carboxyl terminal side of the FNCBD. The collagen-binding physiologically active polypeptide of the present invention is soluble in water and well-adapted for industrial production by bacteria, which is preferably E. coli. Production in yeast, insect cell, or animal cell is also acceptable.

For example, the physiologically active peptide may be ligated on the carboxyl terminal side of the collagen-binding domain of FN, and an appropriate amino acid sequence susceptible to cleavage by a protease (for example, the protease present in living tissues) may be inserted on the amino terminal side of the physiologically active peptide from the hybrid polypeptide to enable release of the physiologically active peptide with no addition of excessive sequence. To be more specific, most proteases cleave carboxyl terminal of the recognition sequence, and when the protease recognition sequence is added on the amino terminal of the physiologically active peptide, no excessive amino acid sequence will be left on the amino terminal of the physiologically active peptide after cleavage by the protease. Such protease recognition sequence may be a newly inserted sequence. However, it is also possible to utilize the C terminal side of the collagen-binding domain for the protease recognition sequence and the cleavage site. In this case, the physiologically active peptide maybe directly ligated to the carboxyl terminal of the collagen-binding domain with no intervening artificial sequence, and no excessive amino acid sequence will be left either on the side of the collagen-binding domain of FN or on the side of the physiological peptide after the cleavage at this side. Matricrine or juxtacrine activity is a mode wherein the physiologically active polypeptide binds to the receptor of a cell while it is immobilized on the solid phase whereupon the cell is subjected to the physiological activity of the polypeptide. When such a mode of activity is preferable, the hybrid polypeptide of the present invention may exhibit its physiological activity by preventing being cleaved. On the other hand, the presence of the protease recognition site is quite important when the physiologically active peptide should be cleaved from the collagen-binding domain of FN and to be released into the liquid phase.

In the collagen-binding physiologically active polypeptide (the hybrid polypeptide) of the present invention wherein the FNCBD and the physiologically active peptide are ligated, both the collagen-binding activity and the physiological activity are maintained at a sufficient level, and therefore, the physiologically active peptide can be retained in a collagen matrix at a high stability and exhibit sustaining physiological activity.

In the collagen-binding physiologically active polypeptide as described above, an amino acid or a peptide may be inserted as a spacer at the site of ligation between the physiologically active peptide and the peptide from fibronectin.

In this case, either the spacer or the spacer and the adjacent sequence may preferably contain protease recognition sequence.

The protease recognition sequence is preferably an enterokinase recognition sequence.

The enterokinase recognition sequence plays the role of a spacer which adjusts the distance between the FNCBD and the physiologically active polypeptide, such as bFGF or the EGF and the enterokinase enables release of the bFGF or the EGF from the FNCBD. Enterokinase is a protease found in duodenal membrane and pancreas of a higher animal. As a consequence, the enterokinase recognition sequence is recognized by the enterokinase to become cleaved, and the bFGF or the EGF is thereby released. Enterokinase is an enzyme which recognizes a sequence of very high specificity, DDDDK to cleave C terminal side of K (Lys), and this sequence is necessary for the investigation of the mechanism how the collagen-binding physiologically active peptide becomes physiologically active and for the industrial production of the collagen-binding physiologically active peptide.

The collagen-binding activity or the collagen-binding ability of the hybridpolypeptide of the present invention is a collagen-binding ability based on the activity of the peptide derived from FN, and not the activity of the physiologically active peptide. Whether the collagen-binding activity is based on FN or the physiologically active peptide maybe confirmed by a competitive inhibition experiment wherein a native FN or a physiologically active peptide is used.

The term “collagen” or the “polypeptide from collagen” used in the present invention designates a native collagen, an immature collagen, an atelocollagen (pepsinized collagen), gelatin (denatured collagen), or a polypeptide constituting such collagen forms.

An application of the fusion polypeptide of the present invention is an agent which enables topical retention or sustained release of the physiologically active polypeptide and a physiological activity-imparting agent containing the collagen-binding physiologically active polypeptide as described above. The present invention also provides such an agent.

A biomaterial exhibiting a controlled physiological activity is also provided. In this biomaterial, the physiologically active peptide is gradually released from the biomaterial by protease cleavage at the site of ligation between the physiologically active peptide and the peptide from FN or at a site in the vicinity of such ligation site.

The biomaterial as described above may be used in a method wherein a cell, a tissue, or an organ is provided with a physiological activity to promote proliferation, differentiation, regeneration, or synthetic activity. The present invention also provides such method.

As a consequence, the present invention also provides, for example, a collagen-binding growth factor and a collagen-binding growth factor/collagen composite which can be used to enable sustained-release or topical retention of the growth factor such as bFGF or EGF at the target site to enhance wound healing after injury or surgery or to promote curing of chronic ulcer or refractory ulcer to the maximum extent. Biological tissues contain a substantial proportion of collagen, and collagen is present in substantially all tissues. Therefore, a physiologically active polypeptide will be retained the tissue at or near the site of administration when the physiologically active polypeptide is imparted the activity of binding to collagen. Then, the topical concentration of the physiologically active polypeptide will be maintained at a high level and the action of the physiologically active polypeptide will be enhanced. Unfavorable side effects of the physiologically active polypeptide caused by the diffusion of the physiologically active polypeptide will be also prevented.

The present invention also provides a method of promoting cell proliferation wherein the collagen-binding physiologically active polypeptide (hybridpolypeptide) is used. To be more specific, a hybrid polypeptide of the present invention wherein the physiologically active peptide is a growth factor can bind to the collagen/gelatin matrix by utilizing its collagen/gelatin-binding activity, and the growth factor will be retained within the matrix without flowing away with the perfusing medium and thereby cell proliferation is promoted.

The present invention is not limited to the use of growth factors such as bFGF and EGF which are effective in wound healing, and various kind of peptides having a wide variety of physiological activities can be employed in the present invention. The present invention has enabled to maintain the physiological activity inherent to the particular physiologically active peptide, and at the same time, the collagen-binding activity imparted to the physiologically active peptide have realized the collagen-binding physiologically active polypeptide that can be used for the sustained-release or topical retention of the physiologically active peptide.

Also provided by the present invention are a gene coding for such collagen-binding physiologically active polypeptide, a recombinant vector including such gene, a transformant having such gene, and a bacterium, preferably E. coli having such vector.

Also provided are a vector for gene therapy wherein the gene coding for the collagen-binding physiologically active polypeptide is incorporated in the vector prepared by using a virus vector such as retrovirus, adenovirus, and adeno-associated vector, or a vector prepared by using liposome method. A cell with a pharmaceutical function wherein such polypeptide or such gene is incorporated is also provided.

A physiologically active peptide/collagen composite matrix which is functionally modified with a physiological activity is provided by combining such hybrid polypeptide with the collagen, and this composite collagen matrix is quite useful as a novel biomaterial for tissue regeneration. To be more specific, the present invention provides a biomaterial comprising a physiologically active peptide/collagen composite which simultaneously has a scaffold property and physiological activity to enable the construction of artificial tissues and organs, having three dimensional structure (bloodvessel, nerve, bone, ear, nose, finger, skin, duodenum, stomach, heart, liver, pancreas, kidney, etc).

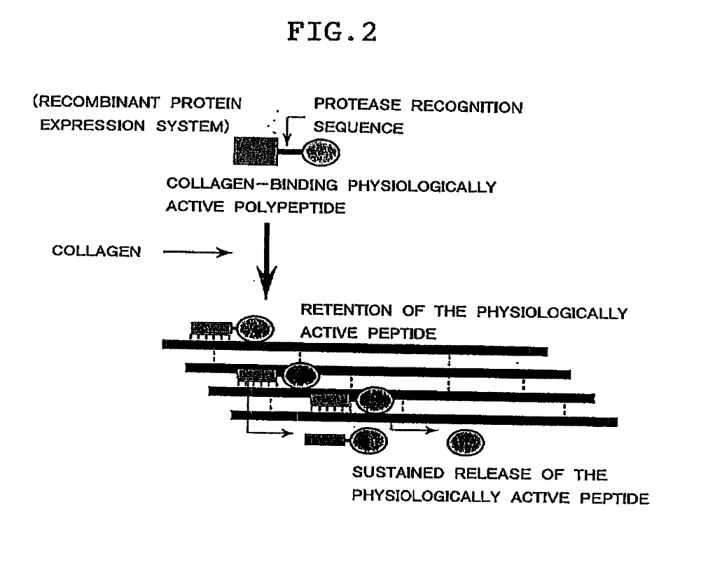

A schematic view of the collagen/physiologically active peptide composite collagen matrix wherein the physiologically active peptide is combined with the collagen matrix is shown in FIG. 2.

Next, the present invention is described by referring to preferred embodiments.

The cDNA and the protein primary structure of the human FN are described in EMBL DATA BANK and The EMBO Journal, vol. 4, No. 7, pages 1755-1759 (1985), respectively. First, the sequence corresponding to the amino acid sequence of the collagen/gelatin-binding domain obtained by limited hydrolysis of the human FN with the protease (plasmin and chymotrypsin) is cloned.

Reverse transcription reaction is conducted by using mRNA extracted from human cells, and the resulting cDNA is subjected to PCR (Polymerase Chain Reaction: Saiki et al., Science, vol. 230, pages 1350 to 1354 (1985)) to amplify the cDNA fragment corresponding to the human FN collagen-binding fragment (FNCBD).

The sense primer used in the PCR has a restriction enzyme recognition sequence and the initiation codon sequence added on its 5′ end, and the antisense primer has a restriction enzyme recognition sequence and antisense sequence of the termination codon added at its 5′ end.

The FNCBD cDNA fragment is then inserted into the cloning vector, pBlueScript SK to constitute plasmid pBS (FNCBD). After confirming the nucleotide sequence, the cDNA fragments is cut out and incorporated in expression vector pTYB1 to construct plasmid pTYB1(FNCBD).

pTYB1(FNCBD) is a plasmid which expresses Ala ²⁶⁰-Trp ⁵⁹⁹ of human FN (340 amino acid residues), and introduction of the pTYB1(FNCBD) into E. coli enables the production of the collagen-binding polypeptide, FNCBD.

The cDNA fragment cloned in plasmid pBS (FNCBD) is also used for the creation of hybridgenes required in the present invention. The cDNA has the initiation codon sequence on its 51 end, and a ligation site, for example, XhoI recognition sequence is introduced immediately before the termination codon added on its 3′ end corresponding to the C terminal of the FNCBD translation region. The ligation of the cDNA of the FNCBD with the cDNA of the physiologically active peptide is enabled by such constitution of the FNCBD cDNA.

The polypeptide of the present invention is prepared by ligating the cDNA of the FNCBD and the cDNA of the physiologically active peptide and expressing the polypeptide by genetic engineering means.

The functional polypeptide of the present invention is, for example, an artificial functional polypeptide wherein a polypeptide of 340 amino acid residues corresponding to Ala ²⁶⁰ -Trp ⁵⁹⁹ of human FN obtained by limited hydrolysis with the protease (plasmin and chymotrypsin) of human FN represented by SEQ ID NO. 1 in the Sequence Listing is ligated with human bFGF or human EGF represented by SEQ ID NO. 2 or SEQ ID NO. 3 in the Sequence Listing.

In the SEQ ID NO. 1 of the Sequence Listing, amino acid No. 1 is Met translated from the initiation codon for expressing the human FNCBD by genetic engineering means; amino acid Nos. 2 to 341 is the amino acid sequence of the human FNCBD; and amino acid Nos. 342 to 343 is Leu and Glu encoded by the XhoI recognition sequence which is used for ligation with the cDNA of the physiologically active peptide. It should be noted that Glu will be removed upon the ligation of the XhoI recognition sequence and SalI recognition sequence.

In the SEQ ID NO. 2 of the Sequence Listing, amino acid No. 1 is Asp encoded by the SalI recognition sequence used for ligation with the cDNA of FNCBD; amino acid Nos. 1 to 5 is enterokinase recognition sequence (Asp-Asp-Asp-Asp-Lys;DDDDK); and amino acid Nos. 6 to 159 is the amino acid sequence of the human bFGF.

SEQ ID NO. 3 of the Sequence Listing, amino acid No. 1 is Asp encoded by the SalI recognition sequence used for ligation with the cDNA of FNCBD; amino acid Nos. 1 to 5 is enterokinase recognition sequence (DDDDK); and amino acid Nos. 6 to 58 is the amino acid sequence of the human EGF.

It should be noted that, the numeral superscripts on the amino acids of the human FN correspond to the number of amino acid residues counted from the N terminal in the mature human FN described in EMBL DATA BANK. The polypeptides comprising 340 amino acid residues corresponding to Ala ²⁶⁰ -Trp ⁵⁹⁹ of human FN shown in SEQ ID NO. 1 in the Sequence Listing differs from the amino acid sequence in EMBL DATA BANK by two amino acid residues, and this difference is not the artificial mutation.

The cDNA and the protein primary structure of the human bFGF are described in Kurokawa et al. , FEBS Letter, vol. 213, No. 1, pages 189-194 (1987). The cDNA and the protein primary structure of the human EGF are described in Bell et al., Nucleic Acids Research, vol. 14, No. 21, pages 8427-8446 (1986).

In the present invention, cDNA of the human bFGF or the human EGF is amplified by PCR from the cDNA prepared by using human mRNA. The PCR of the respective cDNA was conducted by using a sense primer having a restriction enzyme recognition sequence and sequence coding for a protease recognition sequence added at the 5′ end and an antisense primer having a restriction enzyme recognition sequence added at the 5′ end. These cDNA fragments were then inserted in pBluescript SK to prepare pBS (FGF) vector and pBS (EGF) vector, respectively.

The cDNA of the human FNCBD as described above is then ligated to the restriction enzyme recognition sequence on the 5′ end of bFGF or EGF of the plasmid pBS(FGF) or pBS(EGF) to obtain plasmid pBS (FNCBD-FGF) or pBS (FNCBD-EGF) having hybrid genes. The fragment of the hybrid gene is excised from such plasmids and inserted in the expression vector pTYB1 to obtain the recombinant plasmid pTYB1 (FNCBD-FGF) or pTYB1 (FNCBD-EGF) which expresses the hybrid polypeptide represented by SEQ ID NO. 4 or SEQ ID NO. 5 in the Sequence Listing. It should be noted that, in these polypeptides, the human bFGF or the human EGF is ligated to the carboxyl terminal of the human FNCBD.

A nucleotide sequence derived from the PCR primer is present in the hybrid gene between the cDNA of the FNCBD and the cDNA of the bFGF or the EGF. Such sequence will be modified for the adjustment of the molecular distance between the bFGF or the EGF and the FNCBD and/or for the insertion of the protease recognition sequence such as the enterokinase recognition sequence or the like as the spacer peptide. The presence/absence and the type of the protease recognition sequence in the spacer peptide, sequence and length of the spacer peptide, and the like may be selected depending on the purpose of such spacer peptide insertion.

SEQ ID NO. 4 in the Sequence Listing is the amino acid sequence of the hybrid polypeptide of the human FNCBD and human bFGF. Amino acid No. 1 is Met translated from the initiation codon; amino acid Nos. 2 to 341 is the amino acid sequence of the human FNCBD; and amino acid Nos. 342 to 343 is Leu and Asp coded by the nucleotide sequence produced by the ligation of the cDNA of the human FNCBD and the cDNA of the bFGF (ligation of the XhoI recognition sequence and the SalI recognition sequence); amino acid Nos. 343 to 347 is enterokinase recognition sequence (DDDDK); and amino acid Nos. 348 to 501 is the amino acid sequence of the human bFGF.

SEQ ID NO. 5 in the Sequence Listing is the amino acid sequence of the hybrid polypeptide of the human FNCBD and human EGF. Amino acid No. 1 is Met translated from the initiation codon; amino acid Nos. 2 to 341 is the amino acid sequence of the human FNCBD; and amino acid Nos. 342 to 343 is Leu and Asp coded by the nucleotide sequence produced by the ligation of the cDNA of the human FNCBD and the cDNA of the EGF (ligation of the XhoI recognition sequence and the SalI recognition sequence); amino acid Nos. 343 to 347 is enterokinase recognition sequence (DDDDK); and amino acid Nos. 348 to 400 is the amino acid sequence of the human EGF.

When the plasmids pTYB1 (FNCBD), pTYB1 (FNCBD-FGF) and pTYB1 (FNCBD-EGF) constructed as described above are introduced in E. coli, respectively, and cultivated under appropriate conditions, the recombinant polypeptides are accumulated in the E. coli. Such expression may be confirmed by immunoblotting. To be more specific, the proteins from the whole cell of the recombinant E. coli are separated by SDS-polyacrylamide gel electrophoresis, and transferred to a nitrocellulose membrane. The band of the recombinant polypeptides are then detected by using the monoclonal antibodies recognizing each of the FNCBD, the bFGF, and the EGF.

The polypeptide of the present invention is prepared, for example, as described below. The recombinant E. coli having the plasmid introduced therein is cultivated in a culture medium such as SB broth, and IPTG (isopropyl-β-D-galactoside) is added to the culture medium to thereby induce the expression of the gene introduced in the cell. The cells are harvested after further cultivation, and the harvested cells are lysed by sonication. The cell lysate is centrifuged. The recombinant polypeptide in the form of inclusion body is included in the precipitate collected after the centrifugation, and the inclusion body is denatured with 8M urea for solubilization.

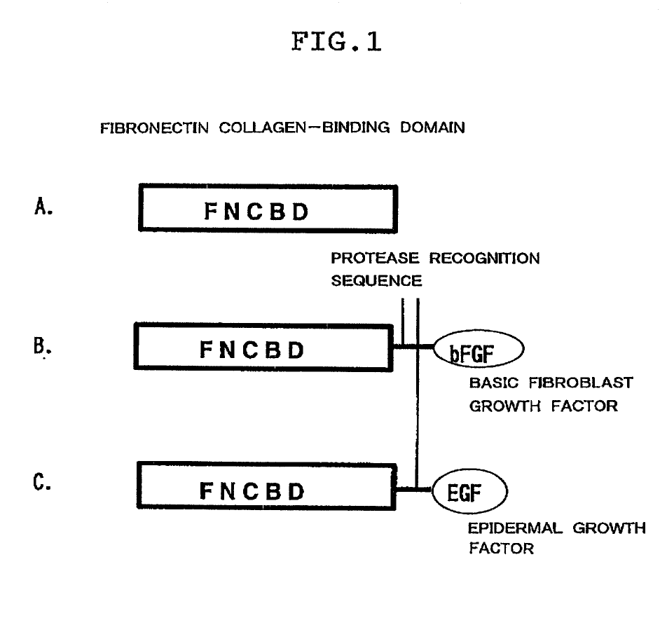

Next, the solubilized inclusion body was dialyzed with the urea concentration incrementally reduced for renaturation of the recombinant protein (refolding treatment). The thus prepared polypeptides of 40 kDa, 57 kDa, and 46 kDa are the embodiments of the FNCBD (FIG. 1, A), the hybrid polypeptide of the FNCBD and the bFGF (FNCBD-FGF; FIG. 1, B), and the hybrid polypeptide of the FNCBD and the EGF (FNCBD-EGF; FIG. 1, C), respectively. B and C are the embodiments of the collagen-binding physiologically active polypeptides.

The collagen-binding activity of the thus prepared polypeptide may be examined by two processes. One is a batchwise process wherein the activity of the polypeptide to bind to gelatin (Gelatin Sepharose 4B, Amersham Pharmacia Biotech) is measured. A mixed suspension of the polypeptide and the Gelatin Sepharose 4B is stirred at 4° C. for 1 hour, and the suspension is subjected to centrifugation. The supernatant is collected, and the precipitate fraction is washed twice with 1M sodium chloride solution and the supernatant is collected. Elution of the polypeptide from gelatin is attempted by repeating the similar procedure with 1M urea, 2M urea, and 4M urea in this order. Finally, the precipitate is boiled in sodium dodecyl sulfate (SDS) buffer, and the supernatant is collected. The collected supernatant fractions are subjected to SDS-polyacrylamide gel electrophoresis (SDS-PAGE), and the binding and elution of the polypeptide to and from gelatin is determined by the presence and the absence of the corresponding band.

In the other process, ability of the FNCBD, the FNCBD-FGF, and the FNCBD-EGF to collagen of different forms, namely, gelatin, native collagen, and atelocollagen (pepsinized collagen) is measured. First, a 96-well plate is coated with gelatin, native collagen, and atelocollagen, and blocked with serum albumin. The FNCBD, the FNCBD-FGF, and the FNCBD-EGF are dispensed, and the plate is incubated at 37° C. for about 1 hour followed by washing and addition of anti-FNCBD monoclonal antibody. After further washing, reaction with a enzyme-conjugated secondary antibody is allowed to take place and the plate was washed. Enzyme substrate is then added for color development, and absorption in the well of the plate is measured as the collagen-binding activity.

Cell-growth promoting activity of the collagen-binding physiologically active polypeptides may be evaluated by XTT method (Roehm et al., Journal Immunological Methods, vol. 142, pages 257 to 265 (1991)) or WST-1 method (Liu et al., Nature Medicine, vol. 1, pages 267 to 271 (1995)) wherein mitochondorial dehydrogenase activity proportion to the number of living cells is measured. To be more specific, mouse BALB/c3T3 cell or human fibroblast is cultivated in a culture medium supplemented with the polypeptide for 4 days. XTT or WST-1 reagent as dehydrogenase substrate is added and the cells are cultivated for further 3 to 6 hours. The culture medium is then measured for its absorption at a particular wavelength to evaluate the cell-growth promoting activity of the particular polypeptide.

The XTT method and the WST-1 method can also be used for the measurement of the cell-growth promoting activity of the collagen matrix combined with the collagen-binding physiologically active polypeptide.

The polypeptide solution of different amount is added to a culture dish coated with collagen, and the solution is allowed to stand at 37° C. for 1 hour. When the polypeptide solution is removed and the dish is extensively washed with physiological solutions, there remains collagen matrix combined with the physiologically active peptide. BALB/c3T3 cell, NRK49F rat fibroblast, or human fibroblast is cultivated on such matrix for 4 days, and for further 3 to 6 hours after adding the XTT or WST-1 reagent. The culture is then measured for its dehydrogenase activity as absorption at a particular wavelength to evaluate the cell-growth promoting activity.

The feature of the present invention, which are believed to be novel are set forth with particularity in the appended claims. The invention, together with objects and advantages thereof, may best be understood by reference to the following description of the presently preferred embodiments and all variation which come within the meaning and range of equivalency of the claims are therefore intended to be embraced therein.

### EXAMPLES

Next, the present invention is described in further detail by referring to the Examples which by no means limit the scope of the invention.

### Example 1

### Preparation of Hybrid Polypeptide of Human FNCBD and Human bFGF, and Hybrid Polypeptide of Human FNCBD and Human EGF

(a) Cloning of the cDNA Coding for Human FNCBD

cDNA of the human FNCBD having a sequence corresponding to the one obtained by limited hydrolysis of the human FN with plasmin and chymotrypsin was prepared by conducing reverse transcription of the mRNA extracted from human kidney cell with the primer (2), and amplifying the resulting cDNA (RT-PCR) with the pair of primers (1) and (2).

In the primer (1) represented by SEQ ID NO. 6 of the Sequence Listing,

nucleotide Nos. 1 to 2 are adjacent sequence added for ensuring KpnI digestion after the PCR,

nucleotide Nos. 3 to 8 are KpnI recognition sequence for the cloning,

nucleotide Nos. 7 to 12, NcoI recognition sequence,

nucleotide Nos. 13 to 14 are the sequence for adjusting the expression frame,

nucleotide Nos. 15 to 20 are NdeI recognition sequence,

nucleotide Nos. 18 to 20 are initiation codon sequence for the expression of the human FNCBD, and

nucleotide Nos. 21 to 49 are nucleotide sequence from the human FNCBD.

In the primer (2) represented by SEQ ID NO. 7 of the Sequence Listing,

nucleotide Nos. 1 to 2 are adjacent sequence added for ensuring BamHI digestion after the PCR,

nucleotide Nos. 3 to 8 are BamHI recognition sequence for the cloning,

nucleotide Nos. 9 to 11 are antisense sequence of termination codon,

nucleotide Nos. 12 to 17 are XhoI recognition sequence for ligation with the cDNA of the bFGF, EGF, or other physiologically active peptides, and

nucleotide Nos. 18 to 46 are antisense sequence from the cDNA of the human FNCBD.

RT-PCR was conducted with RNA LA PCR Kit (AMV) Ver. 1.1 (Takara Shuzo). First, reverse transcription of 0.8 μg of the total RNA was conducted at a reaction solution volume of 20 μl and at a temperature of 60° C. for 20 minutes, and the solution was further heated at 99° C. for 5 minutes.

PCR using the resulting cDNA as the template was then carried out at a reaction solution volume of 100 μl. The solution was maintained at 94° C. for 1 minute, and the temperature cycle of 94° C. for 30 seconds, 63° C. for 1 minutes, and 72° C. for 2 minutes was repeated 12 times.

A 1/10 volume of the reaction solution was analyzed by agarose gel electrophoresis, and there was found a DNA fragment of about 1 kbp. This fragment is of the size corresponding to the cDNA of the human FNCBD.

The thus amplified cDNA fragment was digested with KpnI and BamHI, and ligated to a cloning vector pBluescript SK that had been digested with KpnI and BamHI at 25° C. for 3 minutes (using the ligation kit Ver. 2 manufactured by Takara Shuzo).

Analysis of the nucleotide sequence revealed that a plasmid having incorporated therein the cDNA represented by SEQ ID NO. 8 in the Sequence Listing was obtained. This plasmid was designated pBS(FNCBD).

In SEQ ID NO. 8 of the Sequence Listing,

nucleotide Nos. 1 to 6 are KpnI recognition sequence for the cloning,

nucleotide Nos. 5 to 10 are NcoI recognition sequence,

nucleotide Nos. 11 to 12 are the sequence for adjusting the frame,

nucleotide Nos. 13 to 18 are NdeI recognition sequence,

nucleotide Nos. 16 to 18 are initiation codon sequence for the expression of human FNCBD,

nucleotide Nos. 19 to 1038 are nucleotide sequence of the cDNA of the human FNCBD,

nucleotide Nos. 1039 to 1044 are XhoI recognition sequence for ligation to the cDNA of the bFGF, the EGF, or other physiologically active peptides,

nucleotide Nos. 1045 to 1047 are termination codon, and

nucleotide Nos. 1048 to 1053 are BamHI recognition sequence for the cloning.

It was found that the obtained cDNA sequence of the FNCBD was different from the one registered in EMBL DATA BANK by 5 nucleotides. However, this difference was confirmed not to be caused by the point mutation in the PCR.

(b) Cloning of the cDNA Coding for Human bFGF

cDNA of the human bFGF was prepared by conducing reverse transcription of the mRNA extracted from human kidney cell with the primer (4), and amplifying the resulting cDNA (RT-PCR) with the pair of primers (3) and (4).

In the primer (3) represented by SEQ ID NO. 9 of the Sequence Listing,

nucleotide Nos. 1 to 2 are adjacent sequence added for ensuring SalI digestion after the PCR,

nucleotide Nos. 3 to 8 are SalI recognition sequence for the cloning and for the ligation to the cDNA of the FNCBD,

nucleotide Nos. 6 to 20 are nucleotide sequence coding enterokinase recognition sequence (DDDDK), and

nucleotide Nos. 21 to 40 are nucleotide sequence from the cDNA of the human bFGF.

In the primer (4) represented by SEQ ID NO. 10 of the Sequence Listing,

nucleotide No. 1 is adjacent sequence added for ensuring EcoRI digestion after the PCR,

nucleotide Nos. 2 to 7 are EcoRI recognition sequence for the cloning,

nucleotide Nos. 8 to 10 are antisense sequence of termination codon, and

nucleotide Nos. 11 to 31 are antisense sequence from the cDNA of the human bFGF.

RT-PCR was conducted with RNA LA PCR Kit (AMV) Ver. 1.1 (Takara Shuzo). First, reverse transcription of 0.8 μg of the total RNA was conducted at a reaction solution volume of 20 μl and at a temperature of 60° C. for 20minutes, and the solution was further heated at 99° C. for 5 mitnutes.

PCR reaction was then carried out in 100 μl of the reaction solution containing the cDNA. The solution was maintained at 94° C. for 2minute, and the temperature cycle of 94° C. for 30 seconds and 65° C. for 4 minutes was repeated 30 times.

A 1/10 volume of the reaction solution was analyzed by agarose gel electrophoresis, and there was found a DNA fragment of about 450 bp. This fragment is of the size corresponding to the size of the human bFGF cDNA.

The cDNA fragment was digested with SalI and EcoRI, and ligated to pBluescript SK that had been digested with SalI and EcoRI. A plasmid having incorporated therein the cDNA represented by SEQ ID NO. 11 in the Sequence Listing was obtained. This plasmid was designated pBS(FGF).

In SEQ ID NO. 11 of the Sequence Listing, nucleotide Nos. 1 to 6 are SalI recognition sequence for the cloning and the ligation with the cDNA of the FNCBD, nucleotide Nos. 4 to 18 are nucleotide sequence coding for enterokinase recognition sequence (DDDDK),

nucleotide Nos. 19 to 480 are nucleotide sequence of the cDNA of the human bFGF,

nucleotide Nos. 481 to 483 are termination codon, and

nucleotide Nos. 484 to 489 are EcoRI recognition sequence for the cloning.

It was found that the obtained cDNA sequence of the human bFGF was different from the one registered in EMBL DATA BANK by 1 nucleotide. This difference was confirmed to be the one caused by the point mutation in the PCR. However, this mutation was a silent point mutation with no change in the amino acid, and therefore, the sequence was used with no correction of the nucleotide.

(c) Cloning of the cDNA Coding for Human EGF

cDNA of the human bFGF was prepared by conducing reverse transcription of the mRNA extracted from human kidney cell and with the primer (6), and amplifying the resulting cDNA with the pair of primers (5) and (6).

In the primer (5) represented by SEQ ID NO. 12 of the Sequence Listing,

nucleotide Nos. 1 to 2 are adjacent sequence added for ensuring SalI digestion after the PCR,

nucleotide Nos. 3 to 8 are SalI recognition sequence for the cloning and for the ligation to the cDNA of the FNCBD,

nucleotide Nos. 6 to 20 are nucleotide sequence coding enterokinase recognition sequence (DDDDK), and

nucleotide Nos. 21 to 44 are nucleotide sequence from the cDNA of the human EGF.

In the primer (6) represented by SEQ ID NO. 13 of the Sequence Listing,

nucleotide No. 1 is adjacent sequence added for ensuring EcoRI digestion after the PCR,

nucleotide Nos. 2 to 7 are EcoRI recognition sequence for the cloning,

nucleotide Nos. 8 to 10 are antisense sequence of termination codon, and

nucleotide Nos. 11 to 30 are antisense sequence from the cDNA of the human EGF.

RT-PCR was conducted with RNA LA PCR Kit (AMV) Ver. 1.1 (Takara Shuzo). First, reverse transcription of 1.0 μg of the total RNA was conducted at a reaction solution volume of 20 μl and at a temperature 60° C. for 30 minutes, and the solution was further heated at 99° C. for 5 minutes.

PCR was then carried out at a reaction solution volume of 100 μl. The solution was maintained at 94° C. for 1 minute, and the temperature cycle of 94° C. for 30 seconds and 65° C. for 45 seconds was repeated 35 times.

A 1/10 volume of the reaction solution was analyzed by agarose gel electrophoresis, and there was found a DNA fragment of about 150 bp. This fragment is of the size corresponding to the size of the human EGF cDNA.

The cDNA fragment was digested with SalI and EcoRI, and ligated to the pBluescript SK that had been digested with SalI and EcoRI.

A plasmid having incorporated therein the cDNA represented by SEQ ID NO. 14 in the Sequence Listing was obtained. This plasmid was designated pBS(EGF).

In SEQ ID NO. 14 of the Sequence Listing,

nucleotide Nos. 1 to 6 are SalI recognition sequence for the cloning and the ligation with the cDNA of the FNCBD,

nucleotide Nos. 4 to 18 are nucleotide sequence coding for enterokinase recognition sequence (DDDDK),

nucleotide Nos. 19 to 177 are nucleotide sequence of the cDNA of the human EGF,

nucleotide Nos. 178 to 180 are termination codon, and

nucleotide Nos. 181 to 186 are EcoRI recognition sequence for the cloning.

It should be noted that the obtained cDNA sequence of the human EGF was the same as the one registered in EMBL DATA BANK.

(d) Preparation of the Expression Vector for Human FNCBD

The plasmid pBS (FNCBD) obtained in the above (a) was treated with NdeI and NotI (NotI-recognition sequence is present in the multicloning site of pBluescript SK).

The inserted cDNA fragments of FNCBD was excised, and the fragment was ligated to the NdeI and NotI sites of the expression vector, pTYB1 (New England BioLab). The thus constructed plasmid was designated pTYB(FNCBD).

(e) Preparation of Expression Vector for FNCBD/bFGF Hybrid Polypeptide

The plasmid pBS (FNCBD) obtained in the above (a) was digested with KpnI and XhoI. It should be noted that the plasmid was partially digested since XhoI-recognition sequence was present in the cDNA of the FNCBD produced by RT-PCR of the renal RNA while XhoI-recognition sequence was absent in the sequence registered in EMBL DATA BANK.

The inserted cDNA fragment of the FNCBD was excised by the digestion as described above, and this fragment was ligated to KpnI and SalI sites of the plasmid pBS (FGF). The thus constructed plasmid had incorporated therein the DNA represented by SEQ ID NO. 15 of the Sequence Listing, and this plasmid was designated pBS(FNCBD-FGF).

In SEQ ID NO. 15 of the Sequence Listing, nucleotide Nos. 1 to 6 are KpnI recognition sequence for the cloning,

nucleotide Nos. 5 to 10 are NcoI recognition sequence,

nucleotide Nos. 11 to 12 are the sequence for adjusting the expression frame,

nucleotide Nos. 13 to 18 are NdeI recognition sequence,

nucleotide Nos. 16 to 18 are initiation codon sequence,

nucleotide Nos. 19 to 1038 are cDNA sequence of the human FNCBD.

nucleotide Nos. 1039 to 1044 are the nucleotide sequence formed by the ligation of the XhoI-recognition sequence and the SalI-recognition sequence,

nucleotide Nos. 1042 to 1056 are the nucleotide sequence coding for enterokinase recognition sequence (DDDDK),

nucleotide Nos. 1057 to 1518 are cDNA sequence of the human bFGF,

nucleotide Nos. 1519 to 1521 are termination codon, and

nucleotide Nos. 1522 to 1527 are EcoRI recognition sequence for the cloning.

The DNA fragment of the hybrid gene that had been inserted in pBS (FNCBD-FGF) was cut out with NdeI and EcoRI, and the fragment was ligated to NdeI and EcoRI sites of the expression vector, pTYB1.

The thus constructed plasmid was designated pTYB1 (FNCBD-FGF).

(f) Preparation of Expression Vector for FNCBD/EGF Hybrid Polypeptide

The cDNA fragment of the FNCBD in the plasmid PBS(FNCBD) was excised by treating (partially digesting) with KpnI and XhoI, and the fragment was ligated to KpnI and SalI sites of the plasmid pBS (EGF) obtained in the above (c).

The thus constructed plasmid had incorporated therein the DNA represented by SEQ ID NO. 16 of the Sequence Listing, and this plasmid was designated pBS(FNCBD-EGF).

In SEQ ID NO. 16 of the Sequence Listing,

nucleotide Nos. 1 to 6 are KpnI recognition sequence for the cloning,

nucleotide Nos. 5 to 10 are NcoI recognition sequence,

nucleotide Nos. 11 to 12 are the sequence for adjusting the expression frame,

nucleotide Nos. 13 to 18 are NdeI recognition sequence,

nucleotide Nos. 16 to 18 are initiation codon sequence,

nucleotide Nos. 19 to 1038 are cDNA sequence of the human FNCBD.

nucleotide Nos. 1039 to 1044 are the nucleotide sequence formed by the ligation of the XhoI-recognition sequence and the SalI-recognition sequence,

nucleotide Nos. 1042 to 1056 are the nucleotide sequence coding for enterokinase recognition sequence (DDDDK),

nucleotide Nos. 1057 to 1215 are cDNA sequence of the human EGF,

nucleotide Nos. 1216 to 1218 are termination codon, and

nucleotide Nos. 1219 to 1224 are EcoRI recognition sequence for the cloning.

The DNA fragment of the hybrid gene that had been inserted in pBS (FNCBD-EGF) was cut out with NdeI and EcoRI, and the fragment was ligated to NdeI and EcoRI sites of the expression vector, pTYB1.

The thus constructed plasmid was designated pTYB1 (FNCBD-EGF).

(g) Confirmation of the Expression of the FNCBD/bFGF Hybrid Polypeptide and FNCBD/EGF Hybrid Polypeptide

A strain of E. coli, ER2566 (NEW ENGLAND BioLabs) was transformed with pTYB1(FNCBD), pTYB1(FNCBD-FGF) and pTYB1(FNCBD-EGF), respectively.

The transformed E. coli was cultivated overnight in 2 ml of LB medium supplemented with 100 μg/ml ampicillin at 37° C. 0.02 ml of this preculture medium was inoculated in 2 ml of the SB medium supplemented with 100 μg/ml ampicillin.

After cultivating to a turbidity of 0.5, the culture medium was supplemented with IPTG (isopropyl-β-D-galactocide) to 1 mM, and the cultivation was conducted at 37° C. for another 2 hours. The cells were then harvested.

The protein from the whole cell was subjected to SDS-PAGE (12% gel), and the gel was stained by Coomassie Brilliant Blue. Bands of 40 kDa, 57 kDa, and 46 kDa corresponding to the molecular weight of the FNCBD, the FNCBD/bFGF hybrid, and the FNCBD/EGF hybrid, respectively, were found in the stained gel to confirm the expression of the recombinant polypeptides.

In the meanwhile, the gel after the SDS-PAGE was also transferred to a nitrocellulose membrane, and the membrane was reacted with a monoclonal antibody which specifically recognizes FNCBD (FNC4-4, Takara Shuzo), a monoclonal antibody which specifically recognizes bFGF (FGF-2 (C-18), Santa Cruz Biotechnology), and a monoclonal antibody which specifically recognizes EGF (Clone 10825.1 R&D Systems).

It was then confirmed that the anti-human FNCBD monoclonal antibody reacted with the polypeptide of 40 kDa, that the anti-human FNCBD monoclonal antibody and the anti-human bFGF monoclonal antibody reacted with the polypeptide of 57 kDa, and that the anti-human FNCBD monoclonal antibody and the anti-human EGF monoclonal antibody reacted with the polypeptide of 46 kDa.

These results indicated that the polypeptide of 40 kDa was the human FNCBD, the polypeptide of 57 kDa was the human FNCBD/human bFGF hybrid polypeptide (FNCBD-FGF), and the polypeptide of 46 kDa was the human FNCBD/human EGF hybrid polypeptide (FNCBD-EGF).

The E. coli ER2566 (NEW ENGLAND BioLabs) which had been transformed with the plasmid pTYB1 (FNCBD), pTYB1 (FNCBD-FGF), and pTYB1 (FNCBD-EGF), respectively, and whose expression of the respective polypeptides were confirmed were designated ER2566[pTYB1 (FNCBD)], ER2566[pTYB1 (FNCBD-FGF)], and ER2566[pTYB1 (FNCBD-EGF)] respectively.

(h) Preparation of Expressed Polypeptide

Each of the transformants obtained in the above (g) was cultivated overnight in 2 ml of LB medium supplemented with 100 μg/ml ampicillin at 370C. 0.2 ml of this preculture medium was inoculated in 2 ml of the SB medium supplemented with 100 μg/ml ampicillin. After supplementing with IPTG, the cultivation at 37° C. was conducted and the cells were then harvested.

The thus collected cells were washed with sonication buffer (50 mM Tris-HCl buffer, pH 8.0, 50 mM sodium chloride, 1 mM etylenediamine tetra acetic acid (EDTA)) and centrifuged. The precipitate was suspended in the same buffer again, and the cells were lyzed by repeating 6 cycles of sonication for 15 seconds with interval of 15 seconds.

To this lysate was added TritonX-100 to a final concentration of 1%, and the lysate was centrifuged at 4° C. and at 5,000 rpm for 20 minutes. The precipitate obtained was washed 3 times with an inclusion body-washing solution [0.5% TritonX-100, 1 mM EDTA]. After centrifugation, the insoluble fraction was allowed to solubilize in 8M urea solution [8M urea; S0 mM Tris-HCl buffer, pH 8.0; 1 mM EDTA] at room temperature for 1 hour. Next, this solubilized solution was centrifuged at 4° C. and at 14,000 rpm for 20 minutes to collect the supernatant.

The thus obtained supernatant was dialyzed against 4M urea solution and 2M urea solution in this order, and finally, against sonication buffer or against phosphate buffer [10 mM phosphate buffer, pH 8.0; 50 mM sodium chloride].

After the dialysis, the supernatant was centrifuged at 4° C. and at 14,000 rpm for 20 minutes to prepare the solubilized recombinant protein samples.

The thus obtained samples were subjected to SDS-PAGE, and it was then confirmed that the great majority of each sample was the polypeptide as indicated by the bands at the position of 40 kDa, 57 kDa, or 46 kDa corresponding to the molecular weight calculated from the amino acid sequence of the recombinant protein.

### Example 2

### Evaluation of Biological Activities

The FNCBD, the FNCBD-FGF, and the FNCBD-EGF obtained in Example 1 was evaluated for their collagen-binding activity and cell-growth promoting activity.

(a) Measurement of Collagen-binding Activity

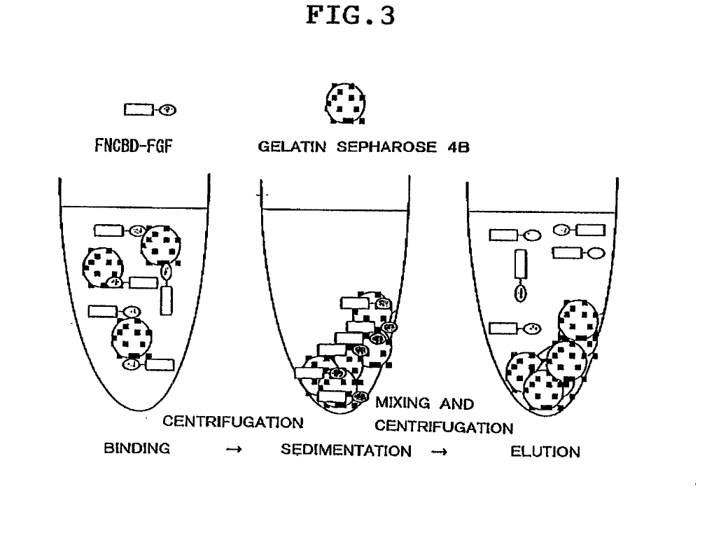

Activity of the FNCBD-FGF to bind to gelatin (Gelatin Sepharose 4B, Amersham Pharmacia Biotech) was evaluated by a batch process as schematically illustrated in FIG. 3.

The recombinant protein sample used was FNCBD-FGF of about 50 μg/ml which had been dialyzed in the Example 1 against sonication buffer.

To a 1.5 ml microtube was added 500 μl of the FNCBD-FGF and 100 μl of the Gelatin Sepharose 4B (which had been washed twice with sonication buffer and prepared as 50% (vol/vol) suspension), and the mixture was stirred at 4° C. for 1 hour.

The mixture was centrifuged at 5000 rpm for 30 seconds and the supernatant was collected. To the precipitate was added 500 μl of 1M sodium chloride solution, and the suspension was centrifuged at 5000 rpm for 30 seconds and the supernatant was collected. In the similar procedure, the precipitate was again washed with 1M sodium chloride solution, and then with 1M urea, 2M urea, and 4M urea solution in this order.

Finally, the suspension was heated in 100 μl SDS sample buffer at 90° C. to elute comlpletely the protein bound to the Gelatin Sepharose 4B.

Ability of the FNCBD-FGF to bind to heparin (AF-Heparin Toyopearl, Tosoh) was also evaluated by a batch process. To 1.5 ml microtube, a mixed suspension of 500 μl of the FNCBD-FGF and 100 μl of AF-Heparin Toyopearl was added (which had been washed twice with sonication buffer and prepared as 50% (vol/vol) suspension), and the suspension was stirred at 4° C. for 1 hour. The suspension was centrifuged at 5000 rpm for 30seconds and the supernatant was collected. The precipitate was washed twice with 500 μl of 1M sodium chloride solution (these supernatants were not subjected to electrophoreses), and heated in 100 μl SDS sample buffer at 90° C. for 5 minutes to elute the bound protein. To 5 μl of each of the supernatant collected, 1 μl of 6 × concentrated SDS sample was added, heated at 90° C. for 5 minutes, and subjected to SDS-PAGE (12% gel).

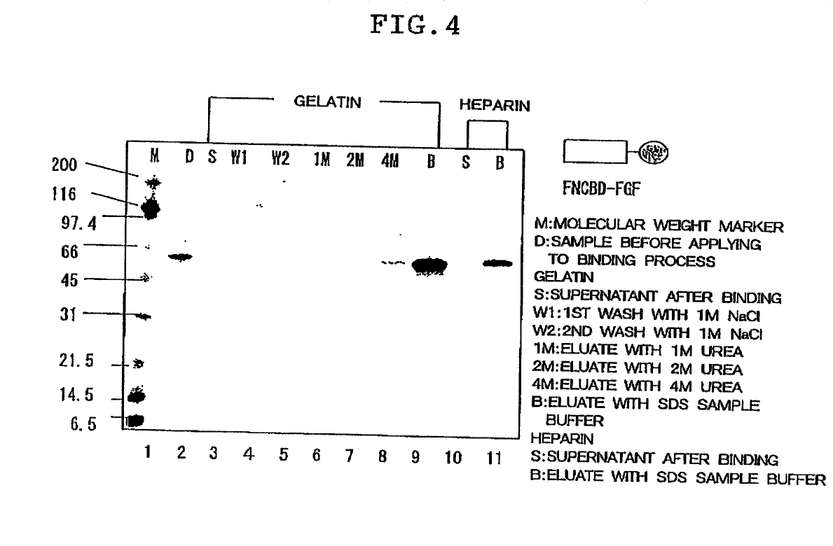

In the SDS-PAGE gel shown in FIG. 4, the band of the FNCBD-FGF around 57 kDa calculated from the amino acid sequence was not detected in lane 3, indicating that most of the FNCBD-FGF bound to gelatin.

The band was not detected in washing solution (lanes 4 and 5), and it was then revealed that the FNCBD-FGF is not eluted from gelatin by the washings with 1M sodium chloride solution.

The band was scarcely recognized in 1M urea and 2M urea eluates (lanes 6 and 7), and a faint band was detected in 4M urea eluate (lane 8).

The FNCBD-FGF band was clearly detected by the elution with SDS buffer at 90° C. (lane 9). In other words, no elution of the FNCBD-FGF was detected with 1 M urea and 2M urea, partial elution was detected with 4M urea, and substantially all of the FNCBD-FGF was eluted with the SDS buffer at 90° C, and strong gelatin-binding activity of the FNCBD-FGF was thereby demonstrated.

In the heparin bindings experiment, the 57 kDa band was not detected in lane 10, suggestion that most of FNCBD-FGF bound to heparin.

The FNCBD-FGF eluted from heparin was detected after heating in SDS buffer at 90° C. (lane 11). Heparin-binding activity of the FNCBD-FGF was thereby demonstrated. In other words, the bFGF moiety in the FNCBD-FGF had a three dimensional structure similar to that of the native bFGF, and the physiological activity of bFGF was suggested to be retained.

As demonstrated above, the strong gelatin-binding activity is retained in the FNCBD moiety of the FNCBD-FGF, and the heparin-binding activity is retained in the bFGF moiety of the FNCBD-FGF.

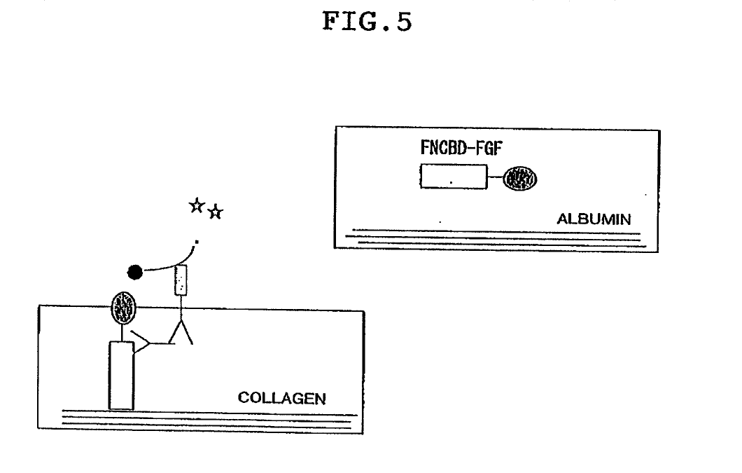

By the enzyme-linked immunosorbent assay (ELISA) as schematically depicated in FIG. 5, binding activity of the FNCBD, the FNCBD-FGF and the FNCBD-EGF to bovine gelatin (Sigma), bovine native collagen (I-AC, Koken), bovine atelocollagen (I-PC, Koken), and bovine serum albumin (BSA, Sigma) was evaluated.

First, 100 μg/ml solutions of gelatin, native collagen, atelocollagen, and serum albumin in phosphate buffered saline (PBS) were respectively dispensed in the wells of a flat-bottom 96 well ELISA plate at 200 μl/well, and the plate was allowed to stand overnight at 4° C.

The solution was discarded and the well was blocked with 3% serum albumin for 1 day at 4° C.

The wells were washed three times with PBS containing 0.05% Tween 20 (PBS-Tween), and 100 μl of the FNCBD, the FNCBD-FGF and the FNCBD-EGF solutions diluted with PBS were respectively dispensed in the well, and the solution was allowed to stand at 37° C. for 1 hour.

The well was washed three times with PBS-Tween, and 100 μl of anti-human FNCBD monoclonal antibody (Takara Shuzo) diluted 1:1000 was dispensed in the well and allowed to stand at room temperature for 1 hour. The well was washed three times with PBS-Tween, and 100 μl of peroxidase-conjugated anti-mouse immunoglobulin polyclonal antibody (Dako Japan) diluted 1:1000 was dispensed in the well and allowed to stand at room temperature for 1 hour. After washing the well for 6 times with PBS-Tween, 0.1M citrate buffer, pH 4.7 containing 1 mg/ml o-phenylenediamine and 0.03% hydrogen peroxide was dispensed in the well and allowed to stand for 10 minutes. The reaction was then terminated by addition of 50 μl of 4 N sulfuric acid and absorption at 492 nm was measured.

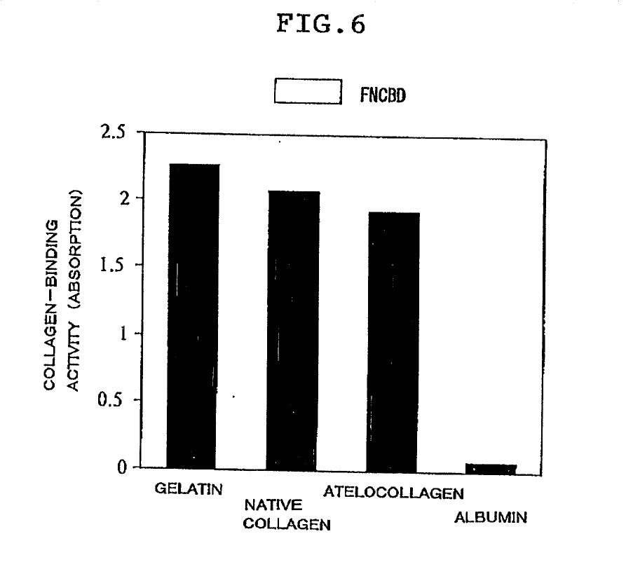

FIG. 6 shows the results when 1 μg/ml FNCBD was reacted with gelatin, native collagen, atelocollagen, or serum albumin immobilized in wells, respectively. When serum albumin was immobilized, the absorption was 0.08, and when gelatin, native collagen, and atelocollagen were immobilized, the absorption was 2.27, 2.08, and 1.93, respectively which were more than 10 times higher than the absorption in the case of serum albumin.

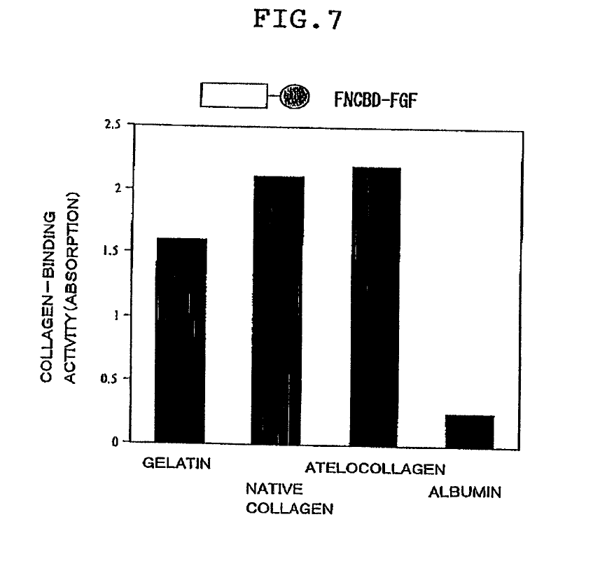

FIG. 7 shows the results when 0.1 μg/ml of FNCBD-FGF was reacted with gelatin, native collagen, atelocollagen, and serum albumin immobilized in wells respectively. Absorption in the case of gelatin, native collagen, and atelocollagen was 1.60, 2.12, and 2.20, respectively, whereas the absorption in the case of serum albumin was 0.27.

As described above, the binding activity of the FNCBD-FGF to gelatin, native collagen, and atelocollagen was markedly higher than the binding activity of the FNCBD-FGF to serum albumin.

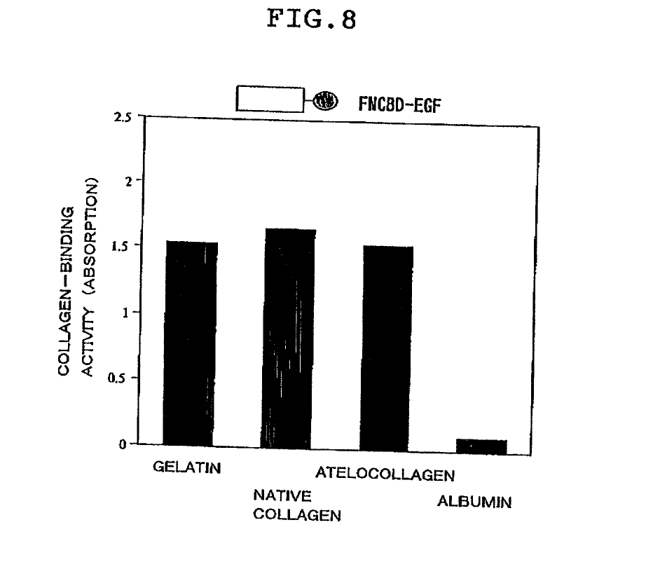

As shown in FIG. 8, the absorption was 0.11, 1.54, 1.66, and 1.54 when the FNCBD-EGF was respectively reacted with serum albumin, gelatin, native collagen, and atelocollagen in a similar manner. The binding activity of the FNCBD-EGF to the immobilized gelatin, native collagen, and atelocollagen was evidently higher than the binding activity of the FNCBD-EGF to serum albumin.

Absorption in ELISA is increased in correlation with the increase in the binding activity. Since absorption after the reaction of the FNCBD with the immobilized gelatin, native collagen, and atelocollagen is about 20 times higher than that after the reaction of the FNCBD with the immobilized serum albumin whose binding may be regarded as a non-specific binding, the reaction of FNCBO with collagen was regarded to be specific reactions (FIG. 6). Similarly, the reaction of the FNCBD-FGF with the immobilized gelatin and collagen as shown in FIG. 7 and the reaction of the FNCBD-EGF with the immobilized gelatin and collagen (FIG. 8) may be regarded as specific reactions. This means that the polypeptide produced in the present invention by genetic engineering means is a molecule wherein the growth factor has been fused with the physiologically active polypeptide with no loss in the collagen binding ability inherent to FNCBD. In summary, it was possible to impart the collagen-binding activity to the growth factor by producing a hybrid polypeptide.

(b) Measurement of Cell-growth Promoting Activity

The FNCBD, the FNCBD-FGF, and the FNCBD-EGF which exhibited collagen-binding activity in the above (a) were also evaluated for their cell-growth promoting activity.

(b-1) Mouse Fibroblast Cell Line

BALB/c3T3 cells (mouse fibroblast cell line) were suspended in Iscove 's Modified Dulbecco's Medium (IMDM) containing 3% fetal bovine serum (FBS), and the suspension was dispensed in 24-well plate at 2×10³ cells/well. The cells were cultivated at 37° C. for 5 hours.

The medium was then changed to IMDM medium containing 0.6% FBS, and the cells were cultivated at 37° C. for another 24 hours.

The FNCBD, the FNCBD-EGF, and the EGF were then added to the cell culture, respectively, and the cultivation was continued at 37° C. for 4 days. The culture medium was supplemented with 1/10 volume of WST-1 reagent (Boeringer Manheim K. K.), and the cultivation was continued at 37° C. for 3 hours.

The culture medium was transferred to a 96-well microtiter plate, and absorption at 450 nm was measured with a microplate reader to evaluate the cell-growth promoting activity.

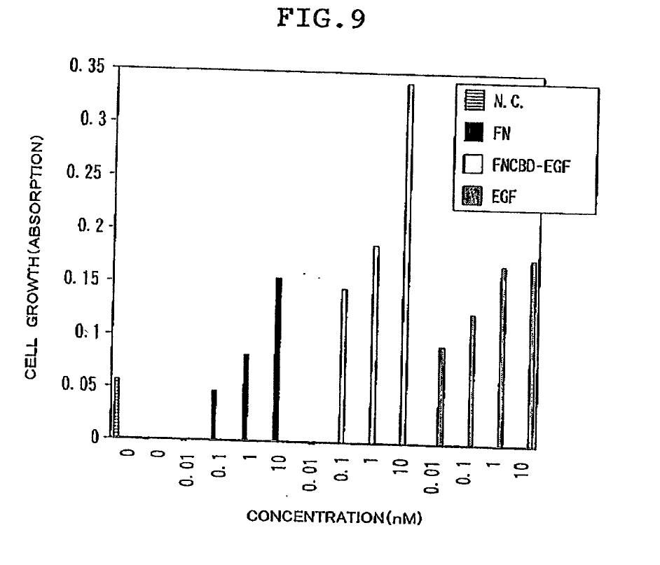

As shown in FIG. 9, the FNCBD-EGF exhibited a concentration-dependent cell-growth promoting activity to the BALB/c3T3 cell, and the activity of the FNCBD-EGF was far beyond that of the EGF. The FNCBD also exhibited some cell-growth promoting activity to BALB/c3T3 cell.

From the results as described above, it may be concluded that the activity of EGF was not reduced by the fusion of the EGF with the FNCBD. It was also indicated that the cell-growth promoting activity of the FNCBD-EGF was the sum of the activity of the FNCBD and that of the EGF from the facts that the FNCBD exhibited some cell-growth promoting activity and that the FNCBD-EGF exhibited the cell-growth promoting activity beyond that of the EGF.

(b-2) Human Fibroblast

Human fibroblast cells were suspended in IMDM containing 3% FBS, and the suspension was dispensed in 24-well plate at 5×10³ cells/well. The cells were cultivated at 37° C. for 5 hours.

The medium was then changed to IMDM containing 0.6% FBS, and the cells were cultivated at 37° C. for another 24 hours.

The FNCBD and the FNCBD-FGF were then added to the cell culture, respectively, and the cultivation was continued at 37° C. for 4 days. The culture medium was supplemented with ½ volume of XTT reagent (Boeringer Manheim K. K.), and the cultivation was continued at 37° C. for 6 hours. The culture medium was transferred to a 96-well microtiterplate, and absorption at 492 nm was measured to evaluate the cell-growth promoting activity.

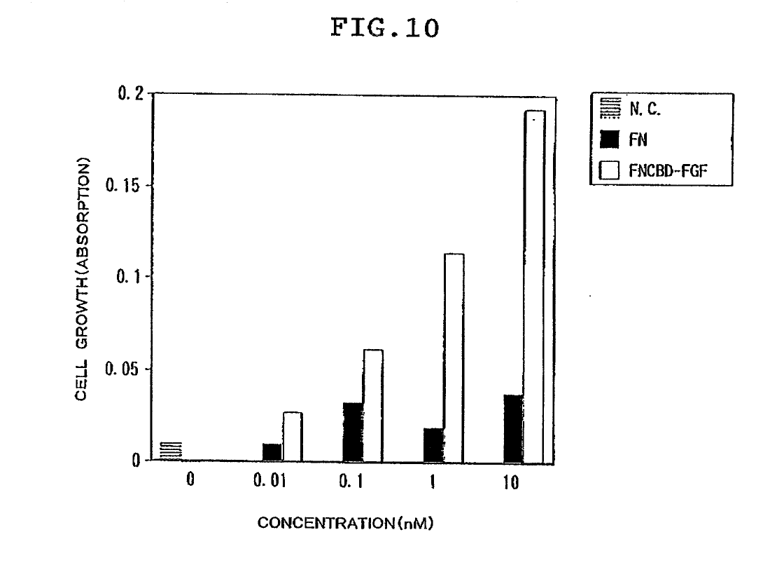

As shown in FIG. 10, the FNCBD-FGF exhibited a concentration-dependent cell-growth promoting activity to the human fibroblast. The FNCBD also exhibited some cell-growth promoting activity to the human fibroblast although the activity was weaker than the case of the BALB/c3T3 cell.

From the results as described above, it may be concluded that the cell-growth promoting activity of bFGF was not diminished while the FGF was endowed with the collagen-binding activity.

(b-3) NRK49F Cell

In the Examples hereinbelow, recombinant protein samples used were those which had been purified with Gelatin Sepharose 4B and dialyzed against phosphate buffer. NRK49F cells (rat fibroblast cell line) were suspended in Dulbecco's Modified Eagle Medium (DMEM) containing 2% FBS, and the suspension was dispensed in 24-well plate at 1×10⁴ cells/well. The cells were cultivated at 37° C. for 7 hours. Serial 5-dilutions of the FNCBD, the FNCBD-EGF, and the EGF were then added to the cell culture (in triplicate wells), respectively, and the cultivation was continued at 37° C. for 4 days. The culture medium was supplemented with 1/10 volume of WST-1reagent (Dojin Kagaku Kenkyujo), and the cultivation was continued at 37° C. for 3 hours. The culture medium was transferred to a 96-well microtiterplate, and absorption at 450 nm was measured to evaluate the cell-growth promoting activity.

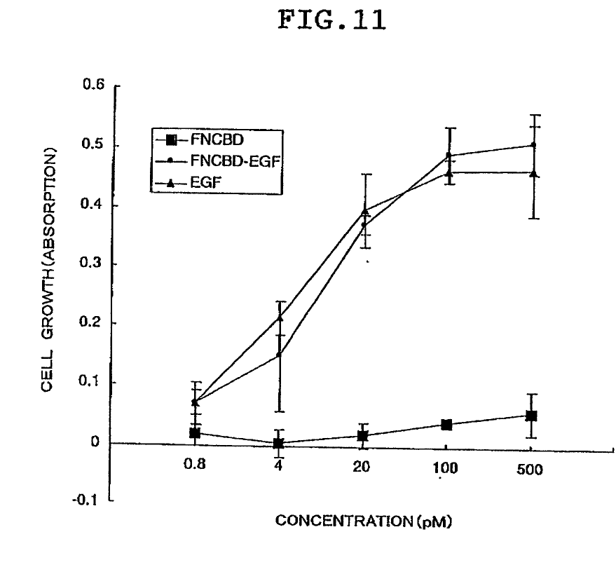

As shown in FIG. 11, the FNCBD-EGF exhibited a concentration-dependent cell-growth promoting activity to the NRK49F, comparable to the cell-growth promoting activity of the EGF. The FNCBD, on the other hand, exhibited no cell-growth promoting activity to the NRK49F cells. Therefore, it may be concluded that the activity of EGF was not lost by the fusion of the EGF with the FNCBD.

### Example 3

### Function of the Hybrid Growth Factor/collagen Composite

Function of the collagen-binding growth factor of the present invention and function of the original growth factor were compared to each other after incubation with collagen.

(a) Cell-growth Promoting Activity of the Growth Factor/collagen Composite

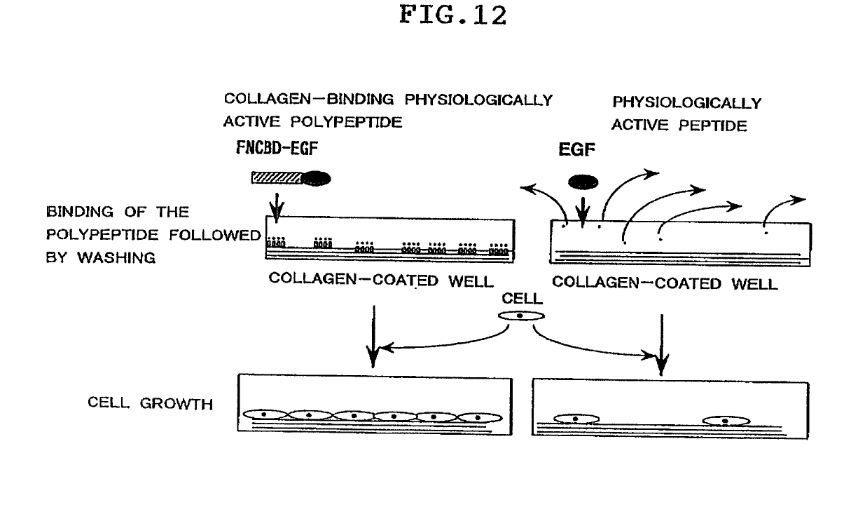

The FNCBD-EGF was used as an example of the collagen-binding physiologically active polypeptide whose collagen-binding activity and cell-growth promoting activity had been confirmed in Examples of 1 and 2. The FNCBD-EGF was evaluated for its cell-growth promoting activity after its binding to collagen (FNCBD-EGF/collagen composite) as schematically depicted in FIG. 12. First, 3 mg/ml solution of atelocollagen in HCl, pH 3.0 was dispensed in the wells of a 24-well plate at 500 l/well, and the solution was allowed to stand overnight at 4° C. The solution was discarded, and wells were washed four times with PBS-Tween, twice with PBS, and once with serum free DMEM. Next, 250 μl of the FNCBD-EGF or EGF solution serially diluted with serum free DMEM (2.5 nM, 5 nM, 10 nM, 20 nM, or 40 nM) was dispensed in the well, and the solution was allowed to, stand at 37° C. for 2 hours. The solution was discarded, and the wells were washed twice with PBS-Tween, 6 times with PBS, and once with Hanks' solution. Human keratinocytes were suspended in serum free human keratinocyte basal medium (KBM2) supplemented with bovine pituitary extract (BPE), and the suspension was then dispensed in the wells of a 24-well plate at 104 cells/0.5 ml/well. If 100% of the FNCBD-EGF or the EGF is retained by the collagen coated on the well, the final concentration will be 1.25 nM, 2.5 nM, 5 nM, 10 nM or 20 nM. The human keratinocytes were cultivated under such conditions at 37° C. for 4days. The culture medium was supplemented with 1/10 volume of WST-1, and the cultivation was continued at 37° C. for 3 hours. The culture medium was transferred to a 96-well microtiterplate, and absorption at 450 nm was measured to evaluate the cell-growth promoting activity.

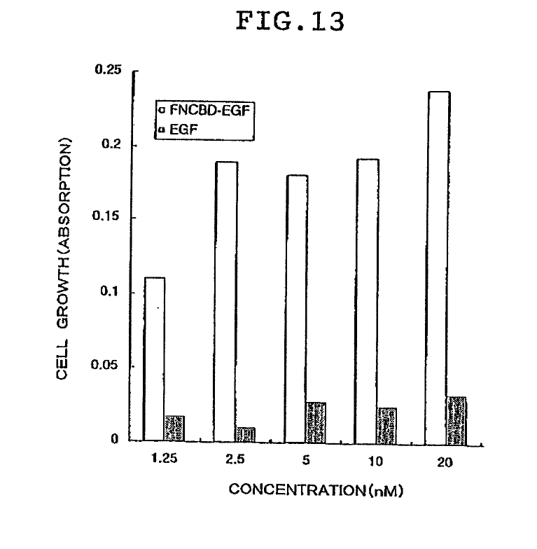

As shown in FIG. 13, the FNCBD-EGF exhibited a concentration-dependent cell-growth promoting activity to human keratinocyte while EGF failed to do so. This difference in the cell-growth promoting activity is judged to be the result of the difference between FNCBD-EGF and the EGF in their collagen-binding abilities. It was thus revealed that the FNCBD-EGF is a collagen-binding physiologically active polypeptide which exhibits the EGF activity after the binding to collagen either by retaining its binding to the collagen or by being gradually released from the collagen.

In addition, the collagen having the FNCBD-EGF bound thereto is an example of the biomaterial which includes both of the collagen-binding EGF and the polypeptide from the collagen (EGF-combined collagen). The method of human keratinocyte cultivation as described above is an example where in a cell is stimulated for its growth activity or other physiological activity.

(b) Comparison between FNCBD-EGF and EGF of Binding Activity to Various Collagen Types

The binding activity of the FNCBD-EGF and the EGF to collagen was compared by using the ELISA. First, wells of a flat bottom 96 well plate were dispensed with 3 mg/ml solution of type I, II, III or IV atelocollagen in HCl (pH 3.0), BSA, or BlockAce (blocking reagent) at 200 μl/well, and the plate was allowed to stand overnight at 4° C. The solution was discarded and the wells were washed 6 times with PBS-Tween. The wells were then supplemented with 100 μl of FNCBD-EGF solution (1.25 nM, 2.5 nM, 5 nM, 10 nM, and 2 nM) or EGF solution serially diluted with serum free DMEM (1.25 nM, 2.5 nM, 5 nM, 10 nM, and 20 nM), respectively, and the wells were allowed to stand at 37° C. for 2 hours. After washing the wells three times with PBS-Tween, 100 μl of anti-human EGF monoclonal antibody diluted 1:1000 with PBS was allowed to stand at room temperature for 1 hour. After washing the wells three times with PBS-Tween, 100 μl of peroxidase-conjugated anti-mouse immunoglobulin polyclonal antibody diluted 1:1000 was dispensed in the wells, and the solution was allowed to stand at room temperature for 1 hour. Finally, the wells were washed 6 times with PBS-Tween, and 0.1M citrate buffer, pH 4.7 containing 1 mg/ml o-phenylenediamine and 0.03% hydrogen peroxide was dispensed to the wells. After allowing to stand for about 10 minutes, the reaction was terminated with 50 μl of 4N sulfuric acid, and the absorption at 492 nm was measured.

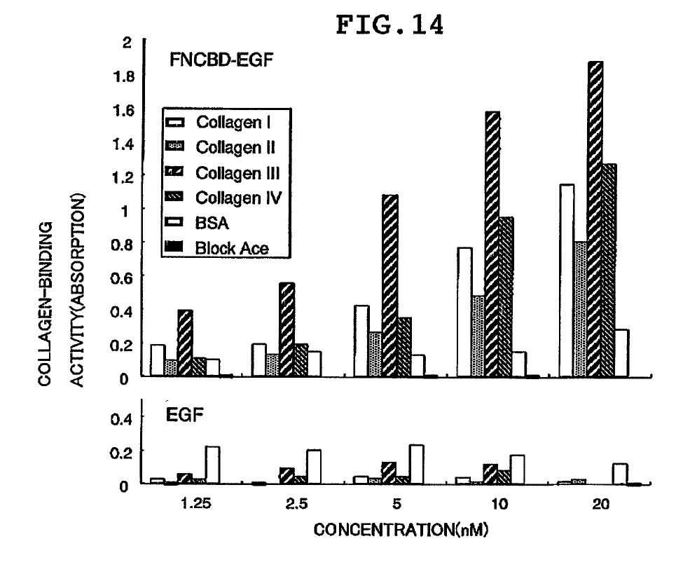

The value of the absorption measured is shown in FIG. 14. The vertical axis represents the collagen-binding activity in terms of the absorption while the concentration of the FNCBD-EGF or the EGF is indicated in the horizontal axis. In the wells coated with type I, II, III, or IV atelocollagen, and incubated with the FNCBD-EGF, the absorption increased in a concentration-dependent manner when the absorption of the well dispensed with no FNCBD-EGF was 0 (control well). In contrast, the absorption of the wells coated with collagens and incubated with the EGF at a concentration from 1.25 nM to 20 nM were substantially the same as the absorption of the control well. Since absorption in ELISA is increased in correlation with the binding activity, it was demonstrated that the FNCBD-EGF wherein the EGF was connected to the collagen-binding domain of FN had a binding activity to various types of collagen markedly higher than that of the original EGF. Therefore, it was confirmed that the activity of promoting the growth of human keratinocyte of the FNCBD-EGF demonstrated in Example 3(a) was mediated by its collagen-binding activity. In other words, FNCBD-EGF is able to exhibit cell-growth promoting activity at the particular site where collagen is immobilized.

As demonstrated in the foregoing examples, retention or sustained release from the site as well as the targeting at the site of collagen has been enabled in the collagen-binding physiologically active peptide of the present invention. It should be noted that the original physiologically active peptide did not have such ability, and such ability has been realized with no sacrifice in its physiological activity by the present invention.

(c) Long Term Stability of the Cell-growth Promoting Activity of the Growth Factor/collagen Composite

The FNCBD-EGF was used as an example of the collagen-binding growth factor whose collagen-binding activity and cell-growth promoting activity had been confirmed. The FNCBD-EGF was evaluated for its cell-growth promoting activity after its binding to collagen (FNCBD-EGF/collagen composite). First, 1 mg/ml solution of atelocollagen in HCl (pH 3.0) was dispensed in the wells of a 24-well plate at 500 μl/well, and the solution was allowed to stand overnight at 4° C. The solution was discarded, and the wells were washed four times with PBS-Tween, twice with PBS, and once with serum free IMEM. Next, 250 μl of the FNCBD-EGF solution diluted with serum free DMEM to 8 pM, 40 pM, 200 pM, and 1000 pM was dispensed in the triplicate wells, and the solution was allowed to stand at 37° C. for 2 hours. The solution was discarded, and the wells were washed twice with PBS, and once with DMEM. The wells were supplemented with DMEM at 500 μl/well, and the solution was allowed to stand at 37° C. for one week. In the meanwhile, 250 μl of the EGF solution diluted with serum free DMEM medium to 8 pM, 40 pM, 200 pM, and 1000 pM was dispensed in the duplicated wells, and the solution was allowed to stand at 37° C. for 2 hours. The solution was discarded, and the wells incubated with the FNCBD-EGF or the EGF were washed twice with PBS-Tween, 6 times with PBS, and once with DMEM. Next, NRK49F rat fibroblast cells were suspended in DMEM containing 2% FBS, and the suspension was dispensed in the wells of a 24-well plate at 10⁴ cells/0.5 ml/well. Assuming 100% of the FNCBD-EGF or the EGF is retained by the collagen coated on the well, the final concentration will be 4 pM, 20 pM, 100 pM and 500 pM. The NRK49F cells were cultivated under such conditions at 37° C. for 4 days. After supplementing the culture medium with 1/10 volume of WST-1 reagent, the cell cultivation was continued at 37° C. for another 3 hours. The culture medium was transferred to a 96-well microtiterplate, and absorption at 450 nm was measured to evaluate the cell-growth promoting activity.

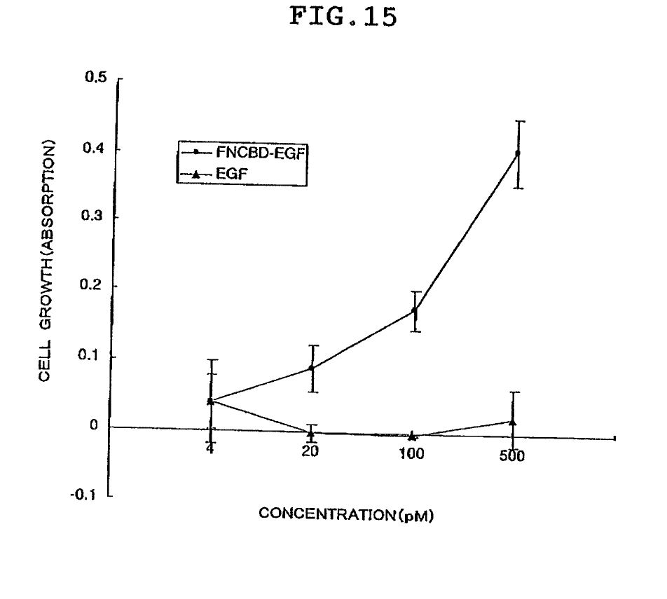

As shown in FIG. 15, the FNCBD-EGF exhibited a concentration-dependent cell-growth promoting activity, while EGF failed to do so. This is judged to be the result of the difference between FNCBD-EGF and the EGF in their collagen-binding ability. It was also revealed that the FNCBD-EGF retains both its collagen-binding activity and EGF activity even after incubation at 37° C. for one week, and that the FNCBD-EGF exhibits its EGF activity after the binding to collagen either by retaining its binding to the collagen or by being gradually released from the collagen. These results indicate that the FNCBD-EGF administered to living tissues is capable of exhibiting its physiological activity to promote growth, differentiation, regeneration or synthetic activity of the cells, tissues, and organs.

(d) Comparison of Collagen-targeting Activity Between FNCBD-EGF and EGF in the Presence of Protein at High Concentration

The targeting activity of the FNCBD-EGF and the EGF to collagen was compared by using the ELISA. First, wells of a flat bottom 96-well plate were supplemented with 3 mg/ml solution of type I atelocollagen in HCl, pH 3.0 at 200 μl/well, and the plate was allowed to stand overnight at 4° C. The solution was discarded and the wells were washed 6 times with PBS-Tween. The wells were then supplemented with 100 μl of FNCBD-EGF solution or the EGF solution diluted with DMEM containing 80mg/ml bovine serum albumin (BSA) to 2 nM, 4 nM, and 8 nM, respectively, and the wells were allowed to stand at 37° C. for 1 hour. After washing the wells three times with PBS-Tween, 100 μl of anti-human EGF monoclonal antibody diluted 1:1000 was dispensed, and the solution was allowed to stand at room temperature for 1 hour. After washing the wells three times with PBS-Tween, 100 μl of peroxidase-conjugated anti-mouse immunoglobulin polyclonal antibody diluted 1:1000 was dispensed in the wells, and the solution was allowed to stand at room temperature for 1 hour. Finally, the wells were washed 6 times with PBS-Tween, and 0.1M citrate buffer, pH 4.7 containing 1 mg/ml o-phenylenediamine and 0.03% hydrogen peroxide was dispensed to the washed wells. After allowing to stand for about 10 minutes, the reaction was terminated with 50 μl of 4N sulfuric acid, and the plate was measured for the absorption at 492 nm.

The value of the absorption measured is shown in FIG. 16. The vertical axis represents the collagen-binding activity in terms of the absorption while the concentration of the FNCBD-EGF or the EGF is indicated in the horizontal axis. Binding of the FNCBD-EGF to collagen increased in a concentration-dependent manner in the presence of BSA at a high concent-reaction. In contrast, the EGF showed substantially no binding to collagen. Consequently, it was demonstrated that the FNCBD-EGF wherein the EGF was connected to the collagen-binding domain of fibronectin had a collagen-targeting activity markedly higher than that of the original EGF in protein solution of a high-concentration. This in turn means that the FNCBD-EGF is capable of specifically targeting the collagen in a tissue where proteins present at high-concentration and capable of exhibiting the cell-growth promoting activity in a topical manner.

As demonstrated in the foregoing examples, retention or sustained release from the site as well as the targeting at the site of collagen has been enabled by the collagen-binding physiologically active peptide of the present invention, and it should be noted the original physiologically active peptide did not have such ability, and such ability has been realized with no sacrifice in its physiological activity. It was also demonstrated that the present invention constitutes a novel drug delivery system.

(e) Influence of Plasma FN on Sustained-release or Topical Retention Properties of the FNCBD-EGF

The sustained-release and release property of the FNCBD-EGF from collagen in the presence of plasma FN was investigated by using the ELISA. First, wells of a flat bottom 96-well plate were supplemented with 3 mg/ml solution of type I or IV atelocollagen in HCl (pH 3.0) at 200 μl/well, and the plate was allowed to stand overnight at 4° C. The solution was discarded and the wells were washed 6 times with PBS-Tween. The wells were then supplemented with 100 μl of FNCBD-EGF solution diluted with DMEM to 20 nM, and the wells were allowed to stand at 37° C. for 90 minutes. After washing the wells three times with PBS-Tween, 100 μl of FN solution diluted with PBS to a concentration of 20, 40, 80, 160, 320, 640, and 1280 nM or with BSA of the same dilution series was dispensed, and the solution was allowed to stand at 37° C. for 90 minutes. After washing the wells three times with PBS-Tween, 100 μl of peroxidase-conjugated anti-mouse immunoglobulin polyclonal antibody diluted 1:1000 was dispensed in the wells, and the solution was allowed to stand at room temperature for 1 hour. Finally, the wells were washed 6 times with PBS-Tween, and 0.1M citrate buffer (pH4.7) containing 1 mg/ml o-phenylenediamine and 0.03% hydrogen peroxide was dispensed. After allowing to stand for about 10 minutes, the reaction was terminated with 50 μl of 4N sulfuric acid, and the plate was measured for the absorption at 492 nm.

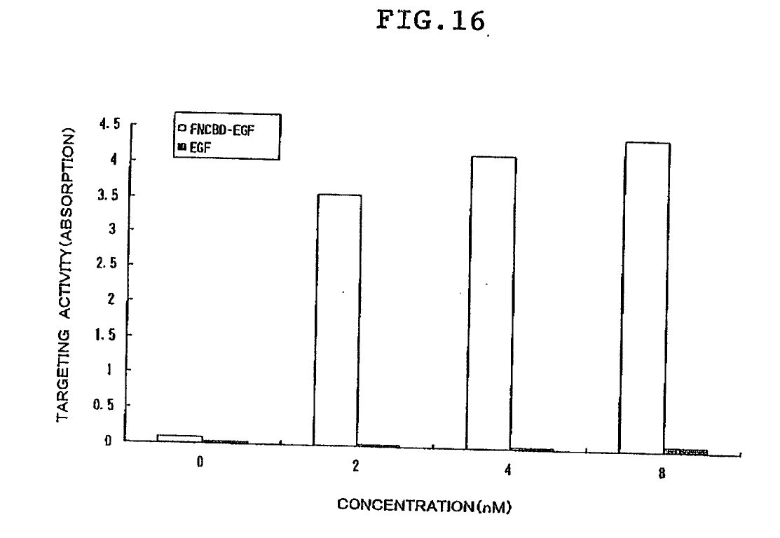

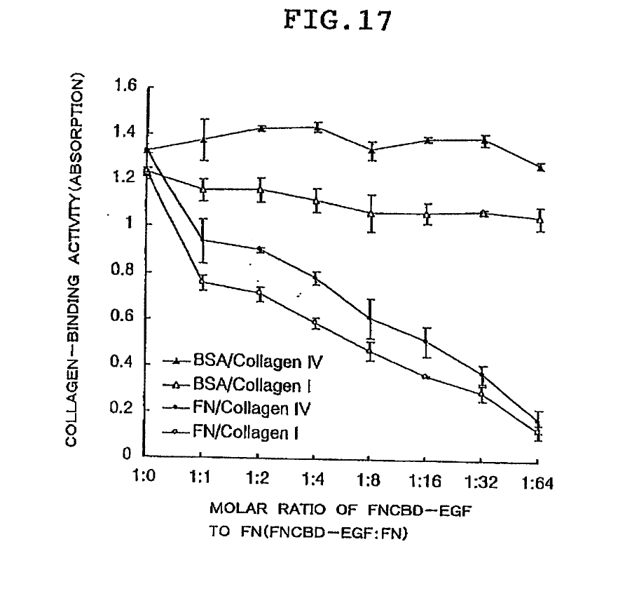

The value of the absorption measured is shown in FIG. 17. The vertical axis represents the collagen-binding activity in terms of the absorption and the concentration of the molar concentration ratio of FNCBD-EGF to FN is indicated in the horizontal axis. In the cases wherein the FN was present at a concentration of 20, 40, 80, 160, 320, 640, and 1280 nM in the wells coated with type I collagen, the value of the absorption decreased in correlation with the concentration of the FN. Since the absorption in ELISA is reduced in correlation with the amount of the FNCBD-EGF bound to the collagen, it was demonstrated that the collagen-binding activity of the FNCBD-EGF wherein the EGF and the collagen-binding domain of fibronectin were fused is competitively inhibited by FN. Therefore, it is highly possible that the FNCBD-EGF that once becomes bound to collagen in the body or at the surface of living body is specifically released by the plasma FN.

As demonstrated in the foregoing examples, the collagen-binding physiologically active polypeptide is gradually released from the collagen when it is exposed to plasma, serum, or blood, or in the co-presence or upon addition of the plasma, serum, or blood, while it is retained in collagen in scarcity of the plasma, serum, or blood, namely, in the scarcity of physiologically active polypeptide such as a growth factor. It was also demonstrated that the present invention constitutes a novel drug delivery system wherein the physiologically active peptide is targeted to collagen and the thus targeted physiologically active peptide undergo controlled retention in the collagen or sustained release from the collagen.

### Example 4

### Preparation of Hybrid Polypeptide of FN485-599/FGF, and Hybrid Polypeptide of FN485-599/EGF

(a) FN485-599/FGF and FN485-599/EGF were Prepared According to the Procedure as Described in Example 1.

Following PCR was carried out by using KOD (TOYOBO Co. LTD.) enzyme.

First, DNA coding for FN485-599 (694-1038of SEQ ID NO. 8in the Sequence Listing) which corresponds to one of proteolytic fragments obtained by limited hydrolysis of human FN with trypsin and chymotrypsin, and exhibits collagen-binding activity, was amplified by PCR with the pTYB1 (FNCBD) as a template using the following primer (7) and the primer (2) represented by SEQ ID NO. 7 of the Sequence Listing. Primer(7):TTCGCATATGGATCAGTGCATTGTTGATGA

A sequence including the Ndel site (TTCGCATATG, underlined is the Ndel site and block letter is the initiation codon) was added at the 5′ end of the amplified DNA by using the primer (7).

After PCR amplification, the Ndel-BamHI fragment of the pBS (FNCBD) was substituted for the Ndel- and BamHI-digested fragment (ca.350 bp) of the PCR amplified DNA, and consequently the plasmid pBS(FN485-599) was constructed.

Next, KpnI-XhoI fragment of the pBS(FN485-599) was ligated to the KpnI and SalI sites of pBS (FGF) or pBS (EGF). Plasmids obtained were designated pBS(FN485-599/FGF) and pBS(FN485-599/EGF) respectively. Ndel-EcoRI fragments of these pBS(FN485-599/FGF) or pBS (FN485-599/EGF) were then inserted between Ndel and EcoRI sites of pTYB 1. The resulting vectors were designated pTYB 1 (FN485-599/FGF) and pTYB1 (FN485-599/EGF) respectively. A host E. coli, ER2566 (NEW ENGLAND BioLabs) was transformed with the vector pTYB1 (FN485-599/FGF) or pTYB1 (FN485-599/EGF), respectively. Transformants were designated respectively ER2566[pTYB1 (FN485-599/FGF)] or ER2566[PTYB 1 (FN485-599/EGF)]. Solubilized recombinant protein samples (each, ca.32 kDa and 21 kDa) were prepared respectively from above transformants and the fusion proteins were designated FN485-599/FGF or FN485-599/EGF.

In the Example hereinbelow, the recombinant protein samples used were those which had been dialyzed finally against phosphate buffer.

(b) Collagen-binding Activity of FN485-599/EGF

The collagen-binding activity of FN485-599/EGF was evaluated for their collagen-binding activity to type I, II, III, IV or V atelocollagen (pepsin-digested collagen) by using the ELISA according to the procedure described as example 3(b).

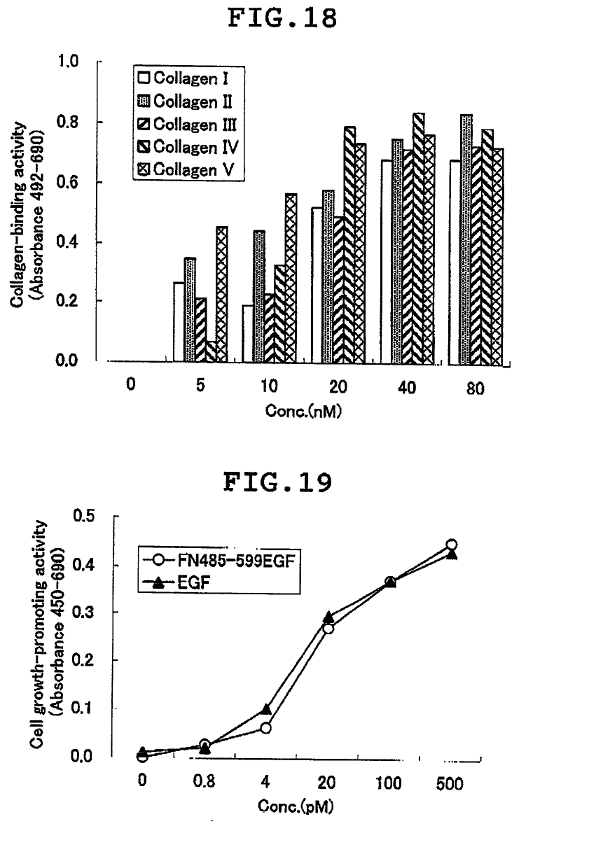

As shown in FIG. 18, the FN485-599/EGF(0, 5, 10, 20, 40, 80 nM) exhibited concentration-dependent collagen-binding activity to type I, II, III, IV or V atelocollagen.

(c) Cell Growth-promoting Activity of FN485-599/EGF

The cell-growth promoting activity of FN485-599/EGF was evaluated according to the procedure described as example 2(b-3).

As shown in FIG. 19, the FN485-599/EGF(0.8, 4, 20, 100, 500 pM) exhibited concentration-dependent cell-growth promoting activity similar to that of a commercial EGF (R&D Systems).

(d) Comparison of Function Between FN485-599/EGF and EGF

EGF Activity of FN485-599/EGF after binding to collagen was evaluated according to the procedure described as example 3(a).

Briefly, NRK49F cells, a DMEM medium, a final concentration of 2%added FBS, and a type I atelocollagen were utillized in this example.

The FN485-599/EGF(4, 20, 100, 500, 2500 pM) also exhibited concentration-dependent cell growth-promoting activity after binding to collagen similar to that of FNCBD-EGF.

As described above, the hybrid protein was prepared by the fusion of the polypeptide comprising FN485-599 (Arg²²⁶ to Trp³⁴¹ of SEQ ID NO. 1 in the Sequence Listing) with FGF or EGF, as a representative instance of the collagen-binding physiologically active polypeptides of the invention. Further, it was demonstrated that this FN485-599/EGF fusion protein exhibited both collagen-binding activity and cell-growth promoting activity, and thus was one of the collagen-binding physiologically active polypeptides of this invention.

Accordingly, it can be substantially deduced that a polypeptide comprising FN485-599(Arg²²⁶ to Trp³⁴¹ of SEQ ID NO. 1) with a physiological peptide such as a cytokine (including a growth factor), a hormonal peptide or an enzyme would be a collagen-binding physiologically active polypeptide which has both collagen-binding activity and physiological activity (inherent activity of above physiological peptide).

### Example 5

### Preparation of Hybrid Polypeptide of FN260-484/FGF, and Hybrid Polypeptide of FN260-484/EGF

(a) FN260-484/FGF and FN260-484/EGF were Prepared According to the Procedure as Described in Example 1.

Following RT-PCR was carried out by using a RNA LAPCR kit (TAKARA) First, DNA (19-693 of SEQ ID NO. 8 in the Sequence Listing) coding for FN260-484 (2-226 of SEQ ID NO. 1 in the Sequence Listing) which corresponds to one of proteolytic fragments obtained by limited hydrolysis of human FN with trypsin, and exhibits collagen-binding activity, was amplified by RT-PCR with RNA of the human mesangial cell as a template using the primer (1) represented by SEQ ID NO. 6 of the Sequence Listing and the following primer (8). Primer(8):GGAATTCTCACTCGAGTCGAAGCTGCGAGTAGGCAATGCATGT

A sequence including the EcoRI site and the XhoI site (GGAATTCTCACTCGAG, underlined are the EcoRI site and the XhoI site, and block letter is the anti-sense sequence of stop codon) was added at the 3′ end of the amplified DNA by using the primer (8).

After RT-PCR amplification, the KpnI and EcoRI digested fragment (ca.700 bp) of the RT-PCR amplified DNA was inserted between the KpnI and EcoRI sites of the pBS, and consequently the plasmid pBS(FN260-484) was constructed.

Next, KpnI-XhoI fragment of the pBS(FN260-484) sized to ca.700 bp by partial digestion with XhoI was ligated to the KpnI and SalI sites of pBS(FGF) or pBS(EGF). Plasmids obtained were designated pBS(FN260-484/FGF) and pBS(FN260-484/EGF) respectively.

Ndel-EcoRI fragments (ca.700 bp) of these pBS(FN260-484/FGF) or pBS (FN260-484/EGF) were then inserted between Ndel and EcoRI sites of pTYB 1. The resulting vectors were designated pTYB 1 (FN260-484/FGF) and pTYB 1 (FN260-484/EGF) respectively.

A host E. coli, ER2566 (NEW ENGLAND BioLabs) was transformed with the vector pTYB 1 (FN260-484/FGF) or pTYB1 (FN260-484/EGF), respectively. Transformants were designated respectively ER2566 [pTYB 1 (FN260-484/FGF)] or ER2566[pTYB 1 (FN260-484/EGF)]. Solubilized recombinant protein samples (each, ca.47 kDa and 36 kDa) were prepared respectively from above transformants and the fusion proteins were designated FN260-484/FGF or FN260-484/EGF.

In the Example hereinbelow, the recombinant protein samples used were those which had been purified with Gelatin and dialyzed finally against phosphate buffer.

(b) Collagen-binding Activity of FN260-484/EGF

The collagen-binding activity of FN260-484/EGF was evaluated for their collagen-binding activity to type I, II or III atelocollagen (pepsin-digested collagen) by using the ELISA according to the procedure described as example 3(b).

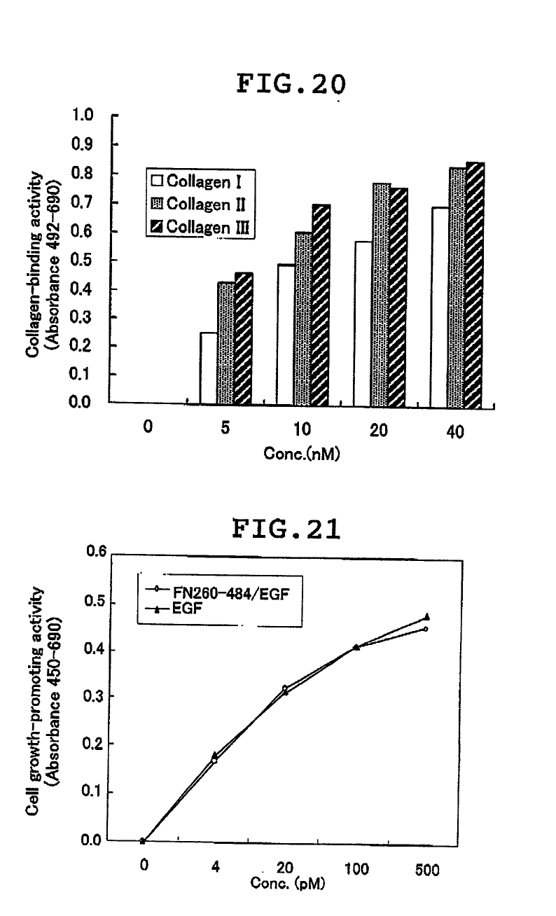

As shown in FIG. 20, the FN260-484/EGF(0, 5, 10, 20, 40 nM) exhibited concentration-dependent collagen-binding activity to type I, II or III atelocollagen.

(c) Cell growth-promoting Activity of FN260-484/EGF

The cell growth-promoting activity of FN260-484/EGF was evaluated according to the procedure described as example 2(b-3).

As shown in FIG. 21, the FN260-484/EGF (4, 20, 100, 500 pM) exhibited concentration-dependent cell-growth promoting activity similar to that of a commercial EGF(R&D Systems).

(d) Comparison of Function Between FN260-484/EGF and EGF

EGF activity of FN260-484/EGF after binding to collagen was evaluated according to the procedure described as example 3(a).

Briefly, NRK49F cells, a DMEM medium, a final concentration of 2% added FBS, and a type I atelocollagen were utillized in this example.

The FN260-484/EGF(4, 20, 100, 500, 2500 pM) also exhibited concentration-dependent cell growth-promoting activity after binding to collagen similar to that of FNCBD-EGF.

As described above, the hybrid protein was the polypeptide comprising FN260-484 (Ala² to Arg²²⁶ of SEQ ID NO. 1 in the Sequence Listing) with FGF or EGF, as a representative instance of the collagen-binding physiologically active polypeptides of the invention.

Further, it was demonstrated that this FN260-484/EGF (Ala² to Arg²²⁶ of SEQ ID NO. 1 in the Sequence Listing) fusion protein exhibited both collagen-binding activity and cell growth-promoting activity, and thus was one of the collagen-binding physiologically active polypeptides of this invention.

Accordingly, it can be substantially deduced that a polypeptide comprising FN260-484(Ala² to Arg²²⁶ of SEQ ID NO. 1) with a physiologically active peptide such as a cytokine (including a growth factor), a hormonal peptide or an enzyme would be a collagen-binding physiologically active polypeptide which has both collagen-binding activity and physiological activity (inherent activity of above physiological peptide).

### MERITS OF THE INVENTION

The collagen-binding physiologically active polypeptide wherein activity of the physiologically active peptide is maintained and binding activity to collagen is imparted is useful in constituting a drug delivery system (DDS) of the physiologically active peptide. Furthermore, the collagen-binding physiologically active polypeptide can be combined with collagen, and the resulting functionally modified collagen matrix is useful as a new biomaterial for use in tissue regeneration.

What is claimed is:

1. A collagen-binding physiologically active polypeptide having both collagen-binding activity and physiological activity different from fibronectin-activities including collagen-binding activity comprising:
a first peptide having collagen-binding activity and consisting of an amino acid sequence which is identical to an amino acid sequence of protease-hydrolysis fragment of fibronectin with at least one protease selected from a group consisting of trypsin, chymotrypsin, thermolysin, plasmin, thrombin, cathepsin D, cathepsin G, pepsin, subtilisin, leukocyte elastase and chymase, and which corresponds to an internal amino acid sequence in collagen-binding domain ranging from 28 kDa to 75 kDa from the amino-terminal of fibronectin,
fused with
a second peptide having physiological activity different from fibronectin activities including collagen-binding activity.

2. A collagen-binding physiologically active polypeptide according to claim 1 wherein said first peptide consists of an internal amino acid sequence of human fibronectin selected from a group consisting of
from Ala²⁶⁰ to Trp⁵⁹⁹ (Ala² to Trp³⁴¹ of SEQ ID NO.1),
from Ala²⁶⁰ to Leu⁴⁸³ (Ala² to Leu ²²⁵ of SEQ ID NO.1),
from Ala²⁶⁰ to Arg⁴⁸⁴ (Ala² to Arg ²²⁶ of SEQ ID NO.1),
from Val²⁶² to Arg⁴⁸⁴ (Val⁴ to Arg ²²⁶ of SEQ ID NO.1),
from Val²⁶² to Trp⁵⁹⁹ (Val⁴ to Trp ³⁴¹ of SEQ ID NO.1),
from Val³⁷⁷ to Leu⁴⁸³ (Val¹¹⁹ to Leu²²⁵ of SEQ ID NO.1),
from Val³⁷⁷ to Trp⁵⁹⁹ (Val 119 to Trp ³⁴¹ of SEQ ID NO.1),
from Leu⁴⁸³ to Trp⁵⁹⁹ (Leu²²⁵ to Trp ³⁴¹ of SEQ ID NO.1),
from Arg⁴⁸⁴ to Trp⁵⁹⁹ (Arg ²²⁶ to Trp ³⁴¹ of SEQ ID NO.1).
from Ala²⁶¹ to Phe⁵⁸⁴ (Ala³ to Phe³²⁶ of SEQ ID NO.1),
from Ala²⁶¹ to Gln⁴⁸² (Ala³ to Gln²²⁴ of SEQ ID NO.1),
from Arg⁴⁸⁴ to Phe⁵⁸⁴ (Arg²²⁶ to Phe³²⁶ of SEQ ID NO.1),
from Val²⁶² to Phe⁵⁸⁴ (Val ⁴ to Phe³²⁶ of SEQ ID NO. 1)
from Leu⁴⁸³ to Phe⁵⁸⁴ (Leu²²⁵ to Phe³²⁶ of SEQ ID NO.1)
and from Asp⁴⁸⁵ to Trp⁵⁹⁹ (Asp ²²⁷ to Trp ³⁴¹ of SEQ ID NO.1).

3. A collagen-binding physiologically active polypeptide ac cording to claim 1 where in said protease-hydrolysis is a proteolysis with a combination of chymotrypsin and plasmin.

4. A collagen-binding physiologically active polypeptide according to claim 3 wherein the first peptide consists of an amino acid sequence of human fibronectin Ala²⁶⁰ to Trp⁵⁹⁹ (Ala² to Trp³⁴¹ of SEQ ID NO.1).

5. A collagen-binding physiologically active polypeptide according to claim 1 wherein said protease-hydrolysis is a proteolysis with trypsin.

6. A collagen-binding physiologically active polypeptide according to claim 5 wherein the first peptide consists an amino acid sequence Ala²⁶⁰ to Arg⁴⁸⁴ (Ala² to Arg²²⁶ of SEQ ID NO.1).

7. A collagen-binding physiologically active polypeptide according to claim 1 wherein said protease-hydrolysis is a proteolysis with a combination of trypsin and chymotrypsin.

8. A collagen-binding physiologically active polypeptide according to claim 7 wherein the first peptide consists of an amino acid sequence Asp⁴⁸⁵ to Trp⁵⁹⁹ (Asp²²⁷ to Trp³⁴¹ SEQ ID NO.1).

9. A collagen-binding physiologically active polypeptide according to claim 1 wherein said second peptide is a physiologically active peptide selected from a group consisting of a cytokine, insulin, parathyroid hormone and matrix metalloproteinases(MMPs).

10. A collagen-binding physiologically active polypeptide according to claim 1 wherein said second peptide is a cytokine.

11. A collagen-binding physiologically active polypeptide according to claim 10 wherein said cytokine is a growth factor.

12. A collagen-binding physiologically active polypeptide according to claim 1 wherein said second peptide is fused on the carboxyl terminal side of said first peptide.

13. A collagen-binding physiologically active polypeptide according to claim 12 wherein an amino acid spacer having less than 7 residues is inserted at the fusion site of the first peptide.

14. A collagen-binding physiologically active polypeptide according to claim 13 wherein a carboxyl terminal of said amino acid spacer is a proteolytic site.

15. A collagen-binding physiologically active polypeptide according to claim 1 wherein said collagen-binding activity is inhibited competitively by fibronectin.

16. A collagen-binding physiologically active polypeptide according to claim 1 wherein said polypeptide is produced in bacteria.

17. A collagen-binding physiologically active polypeptide according to claim 1 wherein said polypeptide is produced in a transformant containing a recombinant vector including the gene coding for said collagen-binding physiologically active polypeptide.

18. An agent for enabling topical retention or sustained release of a physiologically active peptide or a physiological activity-imparting agent which contains the collagen-binding physiologically active polypeptide of claim 1.

19. A biomaterial comprising a composite wherein the collagen-binding physiologically active polypeptide of claim 1 is combined with collagen or gelatin.

20. An agent for enabling topical retention or sustained release of a physiologically active peptide or a physiological activity-imparting agent which contains the biomaterial of claim 19.
